(19) United States
(12) Patent Application Publication
(10) Pub. No.: US 2008/0064856 A1
(43) Pub. Date: Mar. 13, 2008

(54) Methods for reducing protein aggregation

(75) Inventors: Nicholas William Warne, Andover, MA (US); Angela Kantor, Pepperell, MA (US); Thomas Joseph Crowley, Wilmington, MA (US); Erin Christine Soley, Lowell, MA (US); Li Li, Sudbury, MA (US); Nicholas Gary Luksha, Malden, MA (US); Edie Anna Neidhardt, Boxford, MA (US)

Correspondence Address: WilmerHale/Wyeth, 60 STATE STREET, BOSTON, MA, 02109, US

(73) Assignee: Wyeth, Madison, NJ (US)

(21) Appl. No.: 11/725,534

(22) Filed: Mar. 19, 2007

Related U.S. Application Data
(60) Provisional application No. 60/784,130, filed on Mar. 20, 2006.

(51) Int. Cl. C07K 14/755; C07K 1/14; C07K 14/00

(52) U.S. Cl. 530/383; 530/350; 530/412

(57) ABSTRACT

Methods of reducing aggregation of a protein or proteins in a formulation, and protein formulations having reduced aggregation properties are provided. The methods and formulations described herein maintain the biological activity of a protein and increase the shelf life of protein formulations.

### CROSS REFERENCE TO RELATED APPLICATIONS

This application claims the benefit of U.S. Provisional Application No. 60/784,130 filed Mar. 20, 2006, entitled “Methods for Reducing Protein Aggregation,” the contents of which are hereby incorporated by reference in its entirety.

### TECHNICAL FIELD

The field relates to methods of reducing aggregation of proteins and protein formulations that have reduced levels of aggregation.

### BACKGROUND

The completion of the human genome project, coupled with the development of improved methods for protein isolation and purification, have made the large-scale production of protein formulations a reality. In fact, there are more than a hundred recombinant proteins in Phase I clinical trials, or beyond, and several dozen have received Food and Drug Administration approval. Formulations that ensure an efficient and safe delivery of proteins or peptides in a biologically active form are key to the commercial success of current and future biotechnology products.

Unfortunately, proteins possess unique physical and chemical properties, which create difficulties in formulation and development. Physical and chemical instabilities of proteins pose significant challenges in developing suitable protein formulations. The most common physical instability of proteins is protein aggregation and its macroscopic equivalent, precipitation. The tendency of proteins to aggregate is an especially challenging problem in the biotechnology and pharmaceutical industry where it is desired to synthesize, process, and store proteins at the highest possible concentrations, and over long periods of time.

While the mechanisms driving protein aggregation are not completely understood, the end results are nonetheless undesirable. Aggregate formation by a polypeptide in a pharmaceutical composition can adversely affect the biological activity of that polypeptide, resulting in loss of therapeutic efficacy of the pharmaceutical composition. In addition, proteins in an aggregated state can be immunogenic and may even have acute toxic effects in vivo. Furthermore, aggregate formation may cause other problems during administration of the protein formulation, such as blockage of syringes, tubing, membranes, or pumps. Accordingly, there is a need in the art for methods of reducing protein aggregation and for developing protein formulations that exhibit reduced levels of aggregation.

### SUMMARY

This application relates to protein formulations exhibiting reduced aggregation properties and methods of making such formulations.

In one aspect, the application relates to a method for reducing aggregation of a protein or proteins in a formulation by adding methionine to the formulation to a concentration of about 0.5 mM to about 145 mM. The method reduces the aggregation of the protein or proteins in the formulation, compared with the level of aggregation of the same protein or proteins formulated in an identical formulation, except lacking methionine. In a specific embodiment, the method of adding methionine to a formulation to a concentration of about 0.5 mM to about 145 mM reduces the aggregation of the protein or proteins in the formulation when the formulation is subjected to conditions that promote or facilitate protein aggregation, compared with the level of aggregation of the same protein or proteins formulated in an identical formulation, except lacking methionine, and subjected to the same conditions that promote protein aggregation.

In certain embodiments, methionine is added to the formulation to a final concentration of between about 0.5 mM and about 50 mM. In specific embodiments, methionine is added to the formulation to a final concentration of 0.5 mM, 1 mM, 2.5 mM, 5 mM, 7.5 mM, 10 mM, 12.5 mM, 15 mM, 20 mM, 25 mM, 30 mM, 35 mM, 40 mM, and 45 mM. In certain embodiments, the method of adding methionine to a protein formulation to a concentration of about 0.5 mM to about 145 mM, wherein the protein formulation is to be subjected to conditions that lead to protein aggregation, results in a formulation having at most about 5%, at most about 4%, at most about 3%, at most about 2%, at most about 1%, or at most about 0.5% high molecular weight (HMW) species as measured by size exclusion chromatography-high performance liquid chromatography (SEC-HPLC), after the formulation is subjected to conditions that promote protein aggregation.

In some embodiments, the method of adding methionine to a protein formulation to a concentration of about 0.5 mM to about 145 mM increases the shelf life of the protein formulation compared with a formulation lacking methionine. In other embodiments, the method of adding methionine to a protein formulation to a concentration of about 0.5 mM to about 145 mM maintains the potency of the protein formulation compared with a formulation lacking methionine. In certain embodiments, the method of adding methionine to a protein formulation to a concentration of about 0.5 mM to about 145 mM (e.g., about 1 mM to about 145 mM) reduces the immunogenicity of the protein formulation compared with a formulation lacking methionine.

The method is most useful for proteins known to aggregate, or considered likely to aggregate, based on homology to proteins that aggregate, or based on experimental data that suggests the likelihood for aggregation. In one embodiment, the protein within a formulation aggregates during storage. In some embodiments, the protein within a formulation aggregates as a result of shear stress. In other embodiments, the protein within a formulation aggregates as a result of elevated temperature. In other embodiments, the protein within a formulation aggregates as a result of exposure to light. In yet other embodiments, the protein within a formulation aggregates as a result of the presence of certain sugars, or surfactants, in the formulation. The addition of methionine to formulations that are exposed, or likely to be exposed, to such conditions, is effective in reducing aggregate formation, thereby maintaining the biological activity and potency of the protein or proteins within a formulation.

In some embodiments, aggregation of the protein or proteins of the formulation is determined before adding methionine to the formulation. In other embodiments, aggregation of the protein or proteins of the formulation is determined after adding methionine to the formulation. In still further embodiments, aggregation of the protein or proteins of the formulation is determined before and after adding methionine to the formulation. The aggregation of the protein or proteins of a formulation can be determined by any method known to one of ordinary skill in the art including, but not limited to, size exclusion chromatography-high performance liquid chromatography (SEC-HPLC), reverse phase-high performance liquid chromatography (RP-HPLC), UV absorbance, sedimentation velocity measurements, and combinations thereof. In specific embodiments, the percentage high molecular weight (% HMW) species in a formulation comprising about 1 mM to about 145 mM methionine is reduced by at least about 5%, 10%, 15%, 20%, 25%, 30%, 35%, 40%, 45%, 50%, 55%, 60%, 65%, 70%, or 75% compared with % HMW species in the identical formulation, except lacking methionine. In other specific embodiments, a formulation comprising about 1 mM to about 145 mM methionine has at most about 5%, at most about 4%, at most about 3%, at most about 2%, at most about 1%, or at most about 0.5% high molecular weight (HMW) species. Aggregation of a protein or proteins in a formulation can be measured at any time after the formulation is prepared, either with or without methionine. In certain embodiments, aggregation is measured a day after formulating the protein, between 1 week and 12 weeks, or between 1 month and 36 months after formulating the protein of interest.

In some embodiments, the protein of the formulation is an antibody, an immunoglobulin (Ig) fusion protein, a coagulation factor, a receptor, a ligand, an enzyme, a transcription factor, or a biologically active fragment of any of these proteins. In specific embodiments, the protein is an anti-B7.1 antibody, an anti-B7.2 antibody, an anti-CD22 antibody, a PSGL-Ig fusion protein, Factor VIIa, Factor VIII, Factor IX, Factor X, Factor XI, Factor XII, Factor XIII, or a biologically active fragment of any of these proteins. In some embodiments, the protein is formulated at a concentration of from about 0.1 mg/ml to about 250 mg/ml in the formulation. In some embodiments, the protein is formulated at a concentration of from about 0.1 mg/ml to about 200 mg/ml in the formulation. In other embodiments, the protein is formulated at a concentration of from about 0.1 mg/ml to about 100 mg/ml in the formulation. In some embodiments, the protein is formulated at a concentration of from about 0.1 mg/ml to about 10 mg/ml in the formulation. In certain embodiments, the protein is formulated as a liquid or a freeze-dried powder.

In certain embodiments, the protein formulation comprises a surfactant. In specific embodiments, the surfactant is polysorbate-20 or polysorbate-80. In certain other embodiments, the protein formulation lacks a surfactant. In certain embodiments, the protein formulation comprises a tonicity modifier. In specific embodiments, the tonicity modifier is sodium chloride, mannitol, or sorbitol. In certain other embodiments, the protein formulation comprises a sugar. In specific embodiments, the sugar is sucrose, trehalose, mannitol, sorbitol, or xylitol. In certain other embodiments, the protein formulation lacks a sugar. In some embodiments, the pH of the formulation is between about 5.0 and 8.0. In some other embodiments, the pH of the formulation is between about 5.8 and 6.6.

In other embodiments, the protein formulation further comprises one or more agents that reduce aggregation of the protein of the formulation. In some embodiments, the agent that reduces aggregation of the protein of the formulation is an amino acid. In specific embodiments, the amino acid is arginine, lysine, glycine, glutamic acid, or aspartic acid. In some embodiments, the amino acid is added to a protein formulation to a concentration of from about 1 mM to about 300 mM. In some other embodiments, the amino acid is added to a protein formulation to a concentration of from about 5 mM to about 150 mM. In other embodiments, the agent that reduces aggregation of the protein of the formulation is a combination of metal chelators. In specific embodiments, the metal chelators are DTTA, EGTA, and DEF. In some embodiments, the concentration of DTPA or EGTA in the protein formulation is from about 1 mM to about 5 mM. In some embodiments, the concentration of DEF in the protein formulation is from about 1 mM to about 10 mM. In other embodiments, the agent that reduces aggregation of the protein of the formulation is a free radical scavenger, especially a scavenger of oxygen radicals. In specific embodiments, free radical scavenger is mannitol or histidine. In some embodiments, the concentration of mannitol in the protein formulation is from about 0.01% to about 25%. In some embodiments, the concentration of histidine in the protein formulation is from about 100 μM to about 200 mM. In other embodiments, the agent that reduces aggregation of the protein of the formulation is a combination of a metal chelator and a free radical scavenger. In certain other embodiments, the agent that reduces aggregation is citrate. In certain embodiments, the concentration of citrate in the protein formulation is from about 0.5 mM to about 25 mM.

In another aspect, the application provides a method for reducing aggregation of a protein in a protein formulation, wherein the protein does not contain a methionine residue, or contains fewer than 10, 9, 8, 7, 6, 5, 4, 3, or 2 methionine residues, by adding methionine to the formulation to a concentration of about 0.5 mM to about 145 mM. The method results in reduced aggregation of the protein in the formulation compared with the same protein in the identical formulation, except lacking methionine. In certain embodiments, methionine is added to the formulation to a final concentration of between about 0.5 mM and about 50 mM. In specific embodiments, methionine is added to the formulation to a final concentration of 0.5 mM, 1 mM, 2.5 mM, 5 mM, 7.5 mM, 10 mM, 12.5 mM, 15 mM, 20 mM, 25 mM, 30 mM, 35 mM, 40 mM, and 45 mM. In other embodiments, the method of adding about 0.5 mM to about 145 mM methionine to a protein formulation wherein the protein does not contain a methionine residue, or contains fewer than 10, 9, 8, 7, 6, 5, 4, 3, or 2 methionine residues, results in a formulation having at most about 5%, at most about 4%, at most about 3%, at most about 2%, at most about 1%, or at most about 0.5% high molecular weight (HMW) species.

In another aspect, the application provides a method for reducing aggregation of a protein in a protein formulation, wherein the aggregation is not caused by methionine oxidation. The method involves adding methionine to the formulation to a concentration of about 0.5 mM to about 145 mM. The method results in reduced aggregation of the protein in the formulation compared with the same protein in the identical formulation, except lacking methionine. In certain embodiments, methionine is added to the formulation to a final concentration of between about 0.5 mM and about 50 mM. In specific embodiments, methionine is added to the formulation to a final concentration of 0.5 mM, 1 mM, 2.5 mM, 5 mM, 7.5 mM, 10 mM, 12.5 mM, 15 mM, 20 mM, 25 mM, 30 mM, 35 mM, 40 mM, and 45 mM. In other embodiments, the method of adding about 0.5 mM to about 145 mM methionine to a formulation results in a formulation having at most about 5%, at most about 4%, at most about 3%, at most about 2%, at most about 1%, or at most about 0.5% high molecular weight (HMW) species.

In yet another aspect, a method for reducing aggregation of a protein formulated with a surfactant is provided. In certain embodiments, the surfactant causes the protein to aggregate. The method involves adding methionine to the formulation to a concentration of about 0.5 mM to about 145 mM. The method results in reduced aggregation of the protein in the formulation compared with the same protein in the identical formulation, except lacking methionine. In certain embodiments, methionine is added to the formulation to a final concentration of between about 0.5 mM and about 50 mM. In other embodiments, methionine is added to the formulation to a final concentration of 0.5 mM, 1 mM, 2.5 mM, 5 mM, 7.5 mM, 10 mM, 12.5 mM, 15 mM, 20 mM, 25 mM, 30 mM, 35 mM, 40 mM, and 45 mM. In specific embodiments, the method of adding about 0.5 mM to about 145 mM methionine to a formulation formulated with a surfactant results in a formulation having at most about 5%, at most about 4%, at most about 3%, at most about 2%, at most about 1%, or at most about 0.5% high molecular weight (HMW) species.

In a further aspect, a method of adding methionine to a formulation to a concentration of about 0.5 mM to about 145 mM reduces aggregation of a protein subjected to shear stress. The method involves adding the methionine prior to, at the same time as, or after the formulation is subjected to shear stress. The method results in reducing the aggregation of the protein in the formulation compared with the same protein in the identical formulation, except lacking methionine. In certain embodiments, methionine is added to the formulation to a final concentration of between about 0.5 mM and about 50 mM. In specific embodiments, methionine is added to the formulation to a final concentration of 0.5 mM, 1 mM, 2.5 mM, 5 mM, 7.5 mM, 10 mM, 12.5 mM, 15 mM, 20 mM, 25 mM, 30 mM, 35 mM, 40 mM, and 45 mM. In some embodiments, shear stress is caused by agitation, shaking, freeze-thaw, transportation, drawing into a syringe, or purification procedures. In specific embodiments, the method of adding about 0.5 mM to about 145 mM methionine to a formulation subjected to shear stress results in a formulation having at most about 5%, at most about 4%, at most about 3%, at most about 2%, at most about 1%, or at most about 0.5% high molecular weight (HMW) species.

In a still further aspect, a method of adding methionine to a formulation to a concentration of about 0.5 mM to about 145 mM reduces aggregation of a protein exposed to light. In certain embodiments, methionine is added to the formulation to a final concentration of between about 0.5 mM and about 50 mM. In specific embodiments, methionine is added to the formulation to a final concentration of 0.5 mM, 1 mM, 2.5 mM, 5 mM, 7.5 mM, 10 mM, 12.5 mM, 15 mM, 20 mM, 25 mM, 30 mM, 35 mM, 40 mM, and 45 mM. In some embodiments, the light is fluorescent light. In other embodiments, the light is sunlight. In further embodiments, the light is UV light. The method involves adding methionine prior to, at the same time as, or after the formulation is exposed to light. In certain embodiments, methionine is added prior to and at the same time as, or after exposure of the formulation to light. The method of adding methionine to a formulation to a concentration of about 0.5 mM to about 145 mM results in reducing the aggregation of the protein in the formulation compared with the same protein in the identical formulation, except lacking methionine. In specific embodiments, the method of adding about 0.5 mM to about 145 mM methionine to a formulation exposed to light results in a formulation having at most about 5%, at most about 4%, at most about 3%, at most about 2%, at most about 1%, or at most about 0.5% high molecular weight (HMW) species.

In another aspect, a method of adding methionine to a formulation to a concentration of about 0.5 mM to about 145 mM decreases a loss in potency or biological activity of a protein in a protein formulation. This method results in reducing the aggregation of the protein in the formulation, thereby maintaining the potency or functional activity of the protein. In certain embodiments, methionine is added to the formulation to a final concentration of between about 0.5 mM and about 50 mM. In specific embodiments, methionine is added to the formulation to a final concentration of 0.5 mM, 1 mM, 2.5 mM, 5 mM, 7.5 mM, 10 mM, 12.5 mM, 15 mM, 20 mM, 25 mM, 30 mM, 35 mM, 40 mM, and 45 mM. In specific embodiments, the method of adding about 0.5 mM to about 145 mM methionine to a formulation results in a formulation having at most about 5%, at most about 4%, at most about 3%, at most about 2%, at most about 1%, or at most about 0.5% high molecular weight (HMW) species.

In a different aspect, the application provides protein formulations comprising a peptide/peptides, a protein/proteins, or a peptide/peptides and a protein/proteins, and about 0.5 mM to about 50 mM methionine. In specific embodiments, methionine is added to the formulation to a final concentration of 0.5 mM, 1 mM, 2.5 mM, 5 mM, 7.5 mM, 10 mM, 12.5 mM, 15 mM, 20 mM, 25 mM, 30 mM, 35 mM, 40 mM, and 45 mM. In some embodiments of this aspect, the protein of the formulation is an antibody, an Ig fusion protein, a coagulation factor, a receptor, a ligand, an enzyme, a transcription factor, or a biologically active fragment of these proteins. In specific embodiments, the protein is an anti-B7.1 antibody, an anti-B7.2 antibody, an anti-CD22 antibody, a PSGL-Ig fusion protein, Factor VIIa, Factor VIII, Factor IX, Factor X, Factor XI, Factor XII, Factor XIII, or a biologically active fragment of these proteins. In other embodiments, the protein has at least about 85%, at least about 90%, at least about 95%, at least about 96%, at least about 97%, at least about 98%, at least about 99% amino acid sequence identity to an anti-B7.1 antibody, an anti-B7.2 antibody, an anti-CD22 antibody, a PSGL-Ig fusion protein, Factor VIIa, Factor VIII, Factor IX, Factor X, Factor XI, Factor XII, or Factor XIII. In some embodiments, the formulation comprises a buffer. In specific embodiments, the buffer is a histidine buffer, a citrate buffer, a succinate buffer, or a Tris buffer. In certain embodiments, the formulation has a pH of about 5.0 to about 8.0. In other embodiments, the formulation has a pH of about 6.0 to about 7.5. In some embodiments, the formulation comprises another agent that can reduce the aggregation of proteins. The formulation may additionally comprise a sugar, a surfactant, a bulking agent, a cryoprotectant, a stabilizing agent, an anti-oxidant, or a combination of these. In some embodiments, the peptide(s)/protein(s) of the formulation is at a concentration of about 0.1 mg/ml and about 300 mg/ml in the formulation. In other embodiments, the peptide(s)/protein(s) of the formulation is at a concentration of about 0.1 mg/ml and about 10 mg/ml in the formulation. In certain embodiments, the protein is formulated as a liquid, or a freeze-dried powder. In certain embodiments, the protein formulations are provided as kits. Such kits may include buffers, excipients, and instructions for use of the protein formulation.

In another aspect, the application provides methods of treatment, prevention, and/or diagnosis using the protein formulations described herein.

### BRIEF DESCRIPTION OF THE DRAWINGS

FIG. 1a is a bar graph depicting the initial percentage of high molecular weight (% HMW) species in an anti-B7.2 formulation formulated in the presence and absence of 10 mM methionine (Met) and 0.01% polysorbate-80 (PS) at the indicated pH levels.

FIG. 1b is a bar graph depicting the % HMW species in an anti-B7.2 formulation formulated in the presence and absence of 10 mM methionine (Met) and 0.01% polysorbate-80 (PS) at the indicated pH levels, after 6 weeks of storage at 40° C.

FIG. 1c is a bar graph depicting the % HMW species in an anti-B7.2 formulation formulated in the presence and absence of 10 mM methionine (Met) and 0.01% polysorbate-80 (PS) at the indicated pH levels, after 12 weeks of storage at 40° C.

FIG. 2a is a bar graph depicting the initial % HMW species in an anti-B7.1 antibody formulation formulated in citrate, succinate, and histidine buffers (over various pH ranges) in the presence and absence of 10 mM methionine (Met) and 0.01% polysorbate-80 (PS).

FIG. 2b is a bar graph depicting the % HMW species in an anti-B7.1 antibody formulation formulated in citrate, succinate, and histidine buffers (over various pH ranges) in the presence and absence of 10 mM methionine (Met) and 0.01% polysorbate-80 (PS), after 12 weeks of storage at 40° C.

FIG. 3a is a bar graph depicting the % HMW species present in an anti-CD22 antibody formulation after storage for 1 month to 36 months at −80° C.

FIG. 3b is a bar graph depicting the % HMW species present in an anti-CD22 antibody formulation after storage for 1 month to 36 months at 25° C.

FIG. 4 is a graph depicting the % HMW species present in a PSGL-Ig protein formulation, formulated with or without methionine, after storage for up to 4 weeks at −80° C., 25° C., and 40° C.

FIG. 5 is a bar graph depicting the % HMW species in a PSGL-Ig protein formulation subjected to shear stress in the presence (S-1 and S-2) or absence (C) of methionine.

FIG. 6 is a bar graph depicting the potency of REFACTO® formulated in histidine or succinate buffers, with or without methionine, after exposure to light and dark conditions for a period of 1 month.

FIG. 7 is a schematic representation showing the correlation between rhIL-11 oxidation and multimerization.

FIG. 8 provides the amino acid sequences of the light and heavy chains of an anti-B7.1 antibody. The predicted intramolecular disulfide bonds are illustrated by connections of the cysteine residues involved. Cysteines expected to form intermolecular disulfide bonds are underlined and the connectivity indicated. The two altered residues in the Fc portion that reduce effector function are boxed. The N-linked glycosylation consensus site is in bold italics.

FIG. 9 provides the amino acid sequences of the light and heavy chains of an anti-B7.2 antibody. The predicted intramolecular disulfide bonds are illustrated by connections of the cysteine residues involved. Cysteines expected to form intermolecular disulfide bonds are underlined and the connectivity indicated. The two altered residues in the Fc portion that reduce effector function are boxed. The N-linked glycosylation consensus site is in bold italics.

FIG. 10 provides the amino acid sequences of the heavy and light chains of an anti-CD22 antibody. The underlined sequence is the signal sequence and complementarity determining regions are shown in bold letters. A potential site for N-linked glycosylation is underlined.

FIG. 11 provides the amino acid sequence of REFACTO® (see, Sandberg H. et al., Structural and Functional Characterization of B-Domain Deleted Recombinant Factor VIII, Seminars in Hematology, Vol. 38, No. 2, Suppl. 4, pp 4-12, April 2001).

### DETAILED DESCRIPTION

Recent advances in biotechnology have provided a wide variety of biologically active protein formulations for use in diagnosis and therapy. However, the development, production, delivery, safety, and stability of such protein formulations pose significant challenges. One major problem with protein formulations is that they can lose their biological activity as a result of the formation of soluble or insoluble aggregates. Aggregation is a degraded protein state and, therefore, minimizing it results in increased shelf life, potency, or activity of a protein formulation.

This application generally relates to the discovery that the addition of the amino acid methionine to a protein formulation to a final concentration of between about 0.5 mM to about 145 mM, reduces the aggregation of the protein or proteins in the formulation, thereby increasing the shelf-life and maintaining the biological activity of the formulation relative to protein formulations prepared without methionine.

Factors that Affect Protein Aggregation

Proteins have a wide variety of pharmaceutical, biotechnical, and research uses. At various stages in any of these uses, proteins may aggregate. By “aggregate” is meant a physical interaction between protein molecules that results in the formation of covalent or non-covalent dimers or oligomers, which may remain soluble, or form insoluble aggregates that precipitate out of solution. The term “protein,” as used herein, encompasses a peptide, a polypeptide, a protein, and a fusion protein. Proteins may be made by recombinant or synthetic methods.

Many different factors can cause the aggregation of a protein in a protein formulation. Typical purification and storage procedures can expose protein formulations to conditions and components that cause the protein to aggregate. For example, proteins in a protein formulation may aggregate as a result of any one or more of the following: storage, exposure to elevated temperatures, the pH of the formulation, the ionic strength of the formulation, and the presence of certain surfactants (e.g., polysorbate-20 and polysorbate-80) and emulsifying agents. The term “during storage,” as used herein, means a formulation that once prepared, is not immediately used; rather, following its preparation, it is packaged for storage, either in a liquid form, in a frozen state, or in a dried form for later reconstitution into a liquid form or other form. By “elevated temperature” is meant any temperature above the temperature at which the protein is normally stored.

Similarly, proteins may aggregate when exposed to shear stress, such as, reconstituting a lyophilized protein cake in solution, filter-purifying a protein sample, freeze-thawing, shaking, or transferring a protein solution via syringe. Aggregation can also occur as a result of interactions of polypeptide molecules in solution and at the liquid-air interfaces within storage vials. Conformational changes may occur in polypeptides adsorbed to air-liquid and solid-liquid interfaces during compression or extension of the interfaces resulting from agitation during transportation. Such agitation can cause the protein of a formulation to aggregate and ultimately precipitate with other adsorbed proteins.

In addition, exposure of a protein formulation to light can cause the protein to aggregate. Exposure to light can create reactive species that facilitate aggregation. In some embodiments, the light is fluorescent light. In other embodiments, the light is sunlight. In further embodiments, the light is UV light.

Furthermore, the packaging of the protein formulation can impact protein aggregation. Trace levels of metals (ppm levels of copper, iron, cobalt, manganese) can leach out of container packaging, promoting hydrolysis of the amide bond, and ultimately resulting in protein aggregation.

The present application provides methods and compositions that reduce aggregation of proteins by controlling one or more of the above-mentioned aggregation mechanisms. This can result in, for example, improved product stability, and greater flexibility in manufacturing processes and storage conditions.

Methods of Reducing Aggregation of a Protein in a Protein Formulation

This application generally relates to the discovery that adding the amino acid methionine to a formulation can reduce aggregation of a protein or proteins in the formulation. The reduction in aggregation is relative to an identical formulation, except lacking methionine. To reduce aggregation, methionine is added to the formulation to a final concentration of between about 0.5 mM to about 145 mM. As used in this application, “about” means a numeric value having a range of ±25% around the cited value. In some embodiments, methionine is added to a final concentration of between about 0.5 mM to about 10 mM. In other embodiments, methionine is added to a final concentration of between about 0.5 mM to about 15 mM. In some embodiments, methionine is added to a final concentration of between about 2.5 mM to about 10 mM. In some embodiments, methionine is added to a final concentration of between about 2.5 mM to about 15 mM. In other embodiments, methionine is added to a final concentration of between about 5 mM to about 15 mM. In some embodiments, methionine is added to a final concentration of between about 5 mM to about 25 mM. In some other embodiments, methionine is added to a final concentration of between about 0.5 mM to about 25 mM. In certain embodiments, methionine is added to a final concentration of between about 0.5 mM to about 50 mM. In other embodiments, methionine is added to a final concentration of between about 50 mM to about 100 mM. In certain other embodiments, methionine is added to a final concentration of between about 100 mM to about 145 mM. In yet other embodiments, methionine is added to a final concentration of between about 100 mM to about 140 mM. In still other embodiments, methionine is added to a final concentration of between about 100 mM to about 135 mM. In still further embodiments, methionine is added to a final concentration of between about 100 mM to about 125 mM. In other embodiments, methionine is added to a final concentration of between about 5 mM to about 50 mM. In some embodiments, methionine is added to a final concentration of between about 5 mM to about 25 mM. In specific embodiments, methionine is added to a protein formulation to a final concentration of about 0.5 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, or about 50 mM.

Regardless of what causes a protein of a formulation to aggregate, the addition of methionine reduces aggregation of the protein or proteins in the formulation. In certain embodiments, addition of methionine reduces aggregation in a formulation caused by storage, exposure to elevated temperatures, exposure to light, exposure to shear stress, the presence of surfactants, pH and ionic conditions, and any combinations thereof.

The method described above may be used to decrease aggregation of proteins formulated in liquid or dried form. The reduced aggregation is observed in a liquid formulation, whether stored directly in that form for later use, stored in a frozen state and thawed prior to use, or prepared in a dried form, such as a lyophilized, air-dried, or spray-dried form, for later reconstitution into a liquid form or other form prior to use.

The level of protein aggregation in a formulation may be measured before, at substantially the same time as, or after, the addition of methionine to the formulation. In certain embodiments, the level of aggregation is measured at least once between about 1 day and about 12 weeks after the addition of methionine to the formulation. In other embodiments, the level of aggregation is measured at least once between about 1 month and 36 months after the addition of methionine to the formulation. In certain embodiments, the methods described herein result in a reduction of about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of % HMW species compared with formulations lacking methionine. In specific embodiments, the method of adding between about 1 mM to about 145 mM methionine to a protein formulation results in the formulation having at most about 5%, at most about 4%, at most about 3%, at most about 2%, at most about 1%, or at most about 0.5% HMW species. In other specific embodiments, the method of adding between about 1 mM to about 145 mM methionine to a protein formulation results in the formulation having about 5%, about 4%, about 3%, about 2%, about 1%, or about 0.5% HMW species. In other embodiments, the method of adding between about 1 mM to about 145 mM methionine to a protein formulation results in the formulation having between about 0.5% to about 5% HMW species.

The protein formulation may further comprise one or more agents that reduce aggregation of the protein of the formulation. In some embodiments, the agent that reduces aggregation of the protein of the formulation is an amino acid. In specific embodiments, the amino acid is arginine, lysine, glycine, glutamic acid, or aspartic acid. In some embodiments, the amino acid is added to a protein formulation to a concentration of from about 0.5 mM to about 200 mM. In some embodiments, the amino acid is added to a protein formulation to a concentration of from about 5 mM to about 100 mM. In some other embodiments, the amino acid is added to a protein formulation to a concentration of from about 5 mM to about 125 mM.

In certain other embodiments, the amino acid is added to a protein formulation to a concentration of from about 0.5 mM to about 50 mM. In yet other embodiments, the amino acid is added to a protein formulation to a concentration of from about 0.5 mM to about 25 mM. The agent that reduces aggregation of the protein of the formulation can also be a combination of metal chelators. In specific embodiments, the metal chelators are DTPA, EGTA and DEF. In some embodiments, the concentration of DTPA or EGTA in the protein formulation is from about 1 mM to about 10 mM, from about 1 mM to about 5 mM, from about 10 mM to about 10 mM, 50 mM to about 5 mM, or from about 75 μM to about 2.5 mM. In some embodiments, the concentration of DEF in the protein formulation is from about 1 μM to about 10 mM, from about 1 mM to about 5 mM, from about 10 mM to about 1 mM, or from about 20 mM to about 250 μM. The agent that reduces aggregation of the protein of the formulation can also be a free radical scavenger, especially a scavenger of oxygen radicals. In specific embodiments, the free radical scavenger is mannitol or histidine. In some embodiments, the concentration of mannitol in the protein formulation is from about 0.01% to about 25%, from about 0.1% to about 25%, from about 0.5% to about 15%, or from about 1% to about 5%. In some embodiments, the concentration of histidine in the protein formulation is from about 10 μM to about 200 mM, from about 100 μM to about 200 mM, from about 500 μM to about 100 mM, or from about 15 mM to about 35 mM. In other embodiments, the agent that reduces aggregation of the protein of the formulation is a combination of a metal chelator and a free radical scavenger. In some embodiments, the agent that reduces aggregation of a protein or proteins in a formulation is citrate. In certain embodiments, the concentration of citrate in the protein formulation is from about 0.5 mM to about 50 mM, from about 0.5 mM to about 25 mM, from about 1 mM to about 35 mM, from about 5 mM to about 25 mM, or from about 5 mM to about 10 mM.

Methods for Assessing Levels of Protein Aggregation

A number of different analytical methods can be used to detect the presence and levels of aggregates in a protein formulation. These include, but are not limited to, native polyacrylamide gel electrophoresis (PAGE), sodium dodecyl sulfate-polyacrylamide gel electrophoresis (SDS-PAGE), capillary gel electrophoresis (CGE), size exclusion chromatography (SEC), analytical ultracentrifugation (AUC), field flow fractionation (FFF), light scattering detection, sedimentation velocity, UV spectroscopy, differential scanning calorimietry, turbidimetry, nephelometry, microscopy, size exclusion chromatography-high performance liquid chromatography (SEC-HPLC), reverse phase-high performance liquid chromatography (RP-HPLC), electrospray ionization tandem mass spectroscopy (ESI-MS), and tandem RP-HPLC/ESI-MS. These methods may be used either alone, or in combination.

A common problem with protein formulations is the irreversible accumulation of aggregates with time, thermal, or shear stress. Typically, when aggregates precipitate they form large particles that are easy to detect. Smaller, non-covalent soluble aggregates, however, which are often precursors to precipitating large particles are more difficult to detect and quantitate. Thus, methods to detect and quantitate protein aggregation in a protein formulation need to be based on the kind of aggregate being assessed.

Among the above methods, the suggested methods to determine the presence and/or amounts of soluble, covalent aggregates in a protein formulation are: SEC/light scattering, SDS-PAGE, CGE, RP-HPLC/ESI-MS, FFF and AUC. The suggested methods to determine the presence and/or amounts of soluble, non-covalent aggregates in a protein formulation are: SEC, PAGE, SDS-PAGE, CGE, FFF, AUC, and dynamic light scattering. The suggested methods to determine the presence and/or amounts of insoluble, non-covalent aggregates in a protein formulation are: UV spectroscopy, turbidimetry, nephelometry, microscopy, AUC, and dynamic light scattering.

Proteins

Any protein susceptible to aggregation, including antibodies, immunoglobulin fusion proteins, coagulation factors, receptors, ligands, enzymes, transcription factors, or biologically active fragments thereof, can be protected by the methods and compositions of this application. The source or manner in which the protein is obtained or produced (e.g., whether isolated from cells or tissue sources by an appropriate purification scheme, produced by recombinant DNA techniques, or synthesized chemically using standard peptide synthesis techniques) is immaterial to the method taught by this application. Accordingly, a wide variety of native, synthetic, and/or recombinant proteins, including chimeric and/or fusion proteins, can be protected from aggregation by the methods and compositions of this application.

The protein of interest to be formulated includes, but is not limited to, proteins such as, PSGL-Ig; GPIb-Ig; GPIIbIIIa-Ig; IL-13R-Ig; IL-21R-Ig; Factor VIIa; Factor VIII; Factor VIIIC; Factor IX; Factor X; Factor XI; Factor XII; Factor XIII; tissue factor; von Willebrands factor; anti-clotting factors such as Protein C; atrial natriuretic factor; myostatin/GDF-8; interleukins (ILs), e.g., IL-1 to IL-15; human growth hormone and bovine growth hormone; growth hormone releasing factor; parathyroid hormone; thyroid stimulating hormone; uricase; bikunin; bilirubin oxidase; subtilsin; lipoproteins; α-1-antitrypsin; insulin A-chain; insulin B-chain; proinsulin; follicle stimulating hormone; calcitonin; luteinizing hormone; glucagon; lung surfactant; a plasminogen activator, such as urokinase or tissue-type plasminogen activator (t-PA); bombazine; thrombin; plasmin, miniplasmin; microplasmin; tumor necrosis factor-α and -β; enkephalinase; RANTES (regulated on activation normally T-cell expressed and secreted); human macrophage inflammatory protein (MIP-1-α); serum albumin such as human serum albumin; mullerian-inhibiting substance; relaxin A-chain; relaxin B-chain; prorelaxin; mouse gonadotropin-associated peptide; DNase; inhibin; activin; vascular endothelial growth factor (VEGF); placental growth factor (PlGF); receptors for hormones or growth factors; an integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I); insulin-like growth factor binding proteins; CD proteins such as: CD2, CD3, CD4, CD8, CD9, CD19, CD20, CD22, CD28, CD34, and CD45; erythropoietin (EPO); thrombopoietin (TPO); osteoinductive factors; immunotoxins; a bone morphogenetic protein (BMP); an interferon such as interferon-α, -β, and -γ; colony stimulating factors (CSFs), e.g., M-CSF, GM-CSF, and G-CSF; superoxide dismutase; T-cell receptors; members of the HER receptor family such as the EGF receptor, HER2, HER3 or HER4 receptor; cell adhesion molecules such as LFA-1, VLA-4, ICAM-1, and VCAM; IgE; blood group antigens; flk2/flt3 receptor; obesity (OB) receptor; decay accelerating factor (DAF); a viral antigen such as, HIV gag, env, pol, tat, or rev proteins; homing receptors; addressins; immunoadhesins; and biologically active fragments or variants of any of the above-listed polypeptides.

The term “biologically active fragment” means a fragment of a protein that retains at least one of the functions of the protein from which it is derived. A biologically active fragment of an antibody includes an antigen-binding fragment of the antibody; a biologically active fragment of a receptor includes a fragment of the receptor that can still bind its ligand; a biologically active fragment of a ligand includes that portion of a ligand that can still bind its receptor; and a biologically active fragment of an enzyme includes that portion of the enzyme that can still catalyze a reaction catalyzed by the full length enzyme. In certain embodiments, a biologically active fragment retains at least about 25%, 50%, 70%, 75%, 80%, 85%, 90%, or 95% of the function of the protein from which it is derived. The function of a protein can be assayed by well-known methods (e.g., testing antibody-antigen interactions, testing ligand-receptor interactions, testing enzymatic activity, testing transcriptional activity, or testing DNA-protein interactions).

In certain embodiments, the protein to be formulated is an antibody. The antibody may be raised to, and bind to, any of the above-mentioned proteins. In certain specific embodiments, the antibodies include an anti-B7.1 antibody, an anti-B7.2 antibody, an anti-CD22 antibody, an anti-myostatin antibody (e.g., U.S. Appl. No. 60/752,660), an anti-IL-11 antibody, an anti-IL-12 antibody (e.g., U.S. Appl. No. 60/752,660), and an anti-IL-13 antibody (e.g., U.S. Appl. No. 60/752,660). In other specific embodiments, the antibodies include an antibody having at least about 85%, at least about 90%, at least about 95%, at least about 96%, at least about 97%, at least about 98%, or at least about 99% amino acid sequence identity to an anti-B7.1 antibody, an anti-B7.2 antibody, an anti-CD22 antibody, an anti-myostatin antibody (e.g., U.S. Appl. No. 60/752,660), an anti-IL-11 antibody, an anti-IL-12 antibody (e.g., U.S. Appl. No. 60/752,660), or an anti-IL-13 antibody (e.g., U.S. Appl. No. 60/752,660), and retain the ability to bind their respective antigens. Amino acid sequence identity between two proteins can be measured according to standard methods (see, e.g., Pearson and Lipman, Proc. Natl. Acad. Sci. USA 85:2444-2448, 1998; George, D. G. et al., in Macromolecular Sequencing and Synthesis, Selected Methods and Applications pps. 127-149, Alan R. Liss, Inc. 1988; Feng and Doolittle, Journal of Molecular Evolution 25:351-360, 1987; Higgins and Sharp, CABIOS 5:151-153, 1989; and the various BLAST programs of the NCBI, NLM, Bethesda, Md.).

The term “antibody” as used herein, includes polyclonal antibodies, monoclonal antibodies, antibody compositions with polyepitope specificities, bispecific antibodies, diabodies, or other purified preparations of antibodies and recombinant antibodies. The antibodies may be whole antibodies, e.g., of any isotype (IgG, IgA, IgE, IgM, etc.), or fragments thereof, which bind the antigen of interest. In certain embodiments, the antibody to be formulated is an antibody having the IgG isotype.

Recombinant antibodies include, but are not limited to, chimeric and humanized monoclonal antibodies, comprising both human and non-human portions, single-chain antibodies and multi-specific antibodies. A chimeric antibody is a molecule in which different portions are derived from different animal species, such as those having a variable region derived from a murine monoclonal antibody and a human immunoglobulin constant region. Single-chain antibodies have an antigen-binding site and consist of a single polypeptide. Multi-specific antibodies are antibody molecules having at least two antigen-binding sites that specifically bind different antigens. Antibodies can be fragmented using conventional techniques and the fragments screened for binding to the antigen of interest. Preferably, an antibody fragment comprises the antigen-binding and/or the variable region of an intact antibody. Thus, the term antibody fragment includes segments of proteolytically cleaved or recombinantly-prepared portions of an antibody molecule that are capable of selectively binding a certain protein. Non-limiting examples of such proteolytic and/or recombinant fragments include Fab, F(ab′)2, Fab′, Fd, Fv, dAb, an isolated CDR, and single chain antibodies (scFv) containing a V_{L} and/or V_{H} domain joined by a peptide linker. The scFv's may be covalently or noncovalently linked to form antibodies having two or more binding sites.

In some embodiments, the antibody is a humanized monoclonal antibody. The term “humanized monoclonal antibody” as used herein, is a monoclonal antibody from a non-human source (recipient) that has been altered to contain at least one or more of the amino acid residues found in the equivalent human monoclonal antibody (donor). In certain embodiments, the humanized antibodies have one or more complementarity determining regions (CDRs) from the non-human species and a framework region from a human immunoglobulin molecule. A “fully humanized monoclonal antibody” is a monoclonal antibody from a non-human source that has been altered to contain all of the amino acid residues found in the antigen-binding region of the equivalent human monoclonal antibody. Humanized antibodies may also comprise residues that are not found either in the recipient antibody or the donor antibody. These modifications may be made to further refine and optimize antibody functionality. The humanized antibody may also optionally comprise at least a portion of a human immunoglobulin constant region (Fc).

In some embodiments, the protein to be formulated is a fusion protein. In one embodiment, the fusion protein is an immunoglobulin (Ig) fusion protein. An Ig fusion protein is a protein that comprises a non-Ig portion linked to an Ig portion that is derived from the constant region of an immunoglobulin. In a specific embodiment, the fusion protein comprises the IgG heavy chain constant region. In another embodiment, the fusion protein comprises an amino acid sequence corresponding to the hinge, CH2 and CH3 regions of human immunoglobulin Cγ1. Non-limiting examples of Ig fusion proteins include PSGL-Ig (see, U.S. Pat. No. 5,827,817), GPIb-Ig (see, WO 02/063003), GPIIbIIIa-Ig, IL-13R-Ig (see, U.S. Pat. No. 6,268,480), TNFR-Ig (see, WO 04/008100), IL-21R-Ig, CTLA4-Ig and VCAM2D-IgG. Methods of making fusion proteins are well known in the art (e.g., U.S. Pat. Nos. 5,516,964; 5,225,538; 5,428,130; 5,514,582; 5,714,147; 6,136,310; 6,887,471; and 6,482,409). In some embodiments, the proteins of the formulation include fusion proteins having at least about 85%, at least about 90%, at least about 95%, at least about 96%, at least about 97%, at least about 98%, or at least about 99% amino acid sequence identity to PSGL-Ig (see, U.S. Pat. No. 5,827,817), GPIb-Ig (see, WO 02/063003), GPIIbIIIa-Ig, IL-13R-Ig (see, U.S. Pat. No. 6,268,480), TNFR-Ig (see, WO 04/008100), IL-21R-Ig, CTLA4-Ig and VCAM2D-IgG, and which retain their ability to bind their respective ligands.

The formulation may contain more than one protein as necessary for the treatment, or diagnosis of, a particular disease or disorder. The additional protein(s) are chosen because they have complementary activities to the other protein(s) in the formulation, and do not adversely affect the other protein(s) in the formulation. In addition, the protein formulation can also contain non-protein substances that are of use in the ultimate utility of the protein formulation. For example, sucrose can be added to enhance stability and solubility of the protein in solution; and histidine can be added to provide appropriate buffer capacity.

In certain embodiments, the protein to be formulated is essentially pure and/or essentially homogeneous (i.e., substantially free from contaminating proteins, etc). The term “essentially pure” protein means a composition comprising at least about 90% by weight of the protein fraction, preferably at least about 95% by weight of the protein fraction. The term “essentially homogeneous” protein means a composition comprising at least about 99% by weight of the protein fraction, excluding the mass of various stabilizers and water in solution.

The proteins to be formulated may also be conjugated with a cytotoxin, a therapeutic agent, or a radioactive metal ion. In one embodiment, the protein that is conjugated is an antibody or fragment thereof. A cytotoxin or cytotoxic agent includes any agent that is detrimental to cells. Non-limiting examples include, calicheamicin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, and analogs, or homologs thereof. Therapeutic agents include, but are not limited to, antimetabolites (e.g., methotrexate, 6-mercaptopurine, 6-thioguanine, cytarabine, and 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP), cisplatin), anthracyclines (e.g., daunorubicin and doxorubicin), antibiotics (e.g., dactinomycin, bleomycin, mithramycin, and anthramycin), and anti-mitotic agents (e.g., vincristine and vinblastine). Techniques for conjugating such moieties to proteins are well known in the art.

Formulations

The composition of a formulation is determined by consideration of several factors including, but not limited to: the nature of the protein(s) (e.g., receptor, antibody, Ig fusion proteins, enzyme, etc.); the concentration of the protein; the desired pH range; how the protein formulation is to be stored; the period that the protein formulation is to be stored; and whether and how the protein formulation is to be administered to a patient.

Concentration of the Protein in the Formulation

The concentration of the protein in the formulation is dependent on the ultimate use of the protein formulation. Protein concentrations in the formulations described herein are generally between about 0.5 mg/ml and about 300 mg/ml, e.g., between about 0.5 mg/ml and about 25 mg/ml, between about 5 mg/ml and about 25 mg/ml, between about 10 mg/ml and about 100 mg/ml, between about 25 mg/ml and about 100 mg/ml, between about 50 mg/ml and about 100 mg/ml, between about 75 mg/ml and about 100 mg/ml, between about 100 mg/ml and about 200 mg/ml, between about 125 mg/ml and about 200 mg/ml, between about 150 mg/ml and about 200 mg/ml, between about 200 mg/ml and about 300 mg/ml, and between about 250 mg/ml and about 300 mg/ml.

The protein formulations can be used for therapeutic purposes. Accordingly, the concentration of the protein in a formulation is determined based on providing the protein in a dosage and volume that is tolerated by, and of therapeutic value to, the patient. If the protein formulation is to be administered by small volume injection, the protein concentration will be dependent on the injection volume (usually 1.0-1.2 mL). Protein-based therapies usually require several mg/kg of dosing per week, per month, or per several months. Accordingly, if a protein is to be provided at 2-3 mg/kg of body weight of the patient, and an average patient weighs 75 kg, 150-225 mg of the protein will need to be delivered in a 1.0-1.2 mL injection volume, or the volume will need to be increased to accommodate a lower protein concentration.

Buffers

The term “buffer” as used herein, includes those agents that maintain the solution pH in a desired range. The pH of a formulation as described herein is generally between about pH 5.0 to about 9.0, for example, about pH 5.5 to about 6.5, about pH 5.5 to about 6.0, about pH 6.0 to about 6.5, pH 5.5, pH 6.0, or pH 6.5. In general, a buffer that can maintain a solution at pH 5.5 to 6.5 is used. Non-limiting examples of buffers that may be used in a formulation described herein include, histidine, succinate, gluconate, tris (trometamol), Bis-Tris, MOPS, ACES, BES, TES, HEPES, EPPS, ethylenediamine, phosphoric acid, maleic acid, phosphate, citrate, 2-morpholinoethanesulfonic acid (MES), sodium phosphate, sodium acetate, and cacodylate. Histidine is a buffer that is preferred in formulations that are to be administered by subcutaneous, intramuscular, or peritoneal injection. The concentration of the buffer is between about 5 mM and 30 mM. In one embodiment, the buffer of a formulation is histidine at a concentration of about 5 mM to about 20 mM.

Excipients

In addition to the protein, methionine, and buffer, a formulation as described herein may also contain other substances. These substances include, but are not limited to, cryoprotectants, lyoprotectants, surfactants, bulking agents, anti-oxidants, and stabilizing agents. In one embodiment, a protein formulation described herein includes an excipient selected from the group consisting of a cryoprotectant, a lyoprotectant, a surfactant, a bulking agent, an anti-oxidant, a stabilizing agent, and combinations thereof.

The term “cryoprotectant” as used herein, includes agents which provide stability to the protein against freezing-induced stresses, by being preferentially excluded from the protein surface. Cryoprotectants may also offer protection during primary and secondary drying and long-term product storage. Non-limiting examples of cryoprotectants include sugars, such as sucrose, glucose, trehalose, mannitol, mannose, and lactose; polymers, such as dextran, hydroxyethyl starch and polyethylene glycol; surfactants, such as polysorbates (e.g., PS-20 or PS-80); and amino acids, such as glycine, arginine, leucine, and serine. A cryoprotectant exhibiting low toxicity in biological systems is generally used. The cryoprotectant, if included in the formulation, is added to a final concentration of between about 1% and about 10% (weight/volume). In one embodiment, the cryoprotectant is sucrose at a concentration of between about 0.5% and about 10% (weight/volume).

In one embodiment, a lyoprotectant is added to a formulation described herein. The term “lyoprotectant” as used herein, includes agents that provide stability to the protein during the freeze-drying or dehydration process (primary and secondary freeze-drying cycles), by providing an amorphous glassy matrix and by binding with the protein through hydrogen bonding, replacing the water molecules that are removed during the drying process. This helps to maintain the protein conformation, minimize protein degradation during the lyophilization cycle, and improve the long-term product stability. Non-limiting examples of lyoprotectants include sugars, such as sucrose or trehalose; an amino acid, such as monosodium glutamate, non-crystalline glycine or histidine; a methylamine, such as betaine; a lyotropic salt, such as magnesium sulfate; a polyol, such as trihydric or higher sugar alcohols, e.g., glycerin, erythritol, glycerol, arabitol, xylitol, sorbitol, and mannitol; propylene glycol; polyethylene glycol; pluronics; and combinations thereof. The amount of lyoprotectant added to a formulation is generally an amount that does not lead to an unacceptable amount of degradation/aggregation of the protein when the protein formulation is lyophilized. Where the lyoprotectant is a sugar (such as sucrose or trehalose) and the protein is an antibody, non-limiting examples of lyoprotectant concentrations in the protein formulation are from about 10 mM to about 400 mM, and preferably from about 30 mM to about 300 mM, and most preferably from about 50 mM to about 100 mM.

In certain embodiments, a surfactant may be included in the formulation. The term “surfactant” as used herein, includes agents that reduce the surface tension of a liquid by adsorption at the air-liquid interface. Examples of surfactants include, without limitation, nonionic surfactants, such as polysorbates (e.g., polysorbate 80 or polysorbate 20); poloxamers (e.g., poloxamer 188); Triton™; sodium dodecyl sulfate (SDS); sodium laurel sulfate; sodium octyl glycoside; lauryl-sulfobetaine, myristyl-sulfobetaine, linoleyl-sulfobetaine, stearyl-sulfobetaine, lauryl-sarcosine, myristyl-sarcosine, linoleyl-sarcosine, stearyl-sarcosine, linoleyl-betaine, myristyl-betaine, cetyl-betaine, lauroamidopropyl-betaine, cocamidopropyl-betaine, linoleamidopropyl-betaine, myristamidopropyl-betaine, palmidopropyl-betaine, isostearamidopropyl-betaine (e.g., lauroamidopropyl), myristamidopropyl-, palmidopropyl-, or isostearamidopropyl-dimethylamine; sodium methyl cocoyl-, or disodium methyl ofeyl-taurate; and the Monaquat™ series (Mona Industries, Inc., Paterson, N.J.), polyethyl glycol, polypropyl glycol, and copolymers of ethylene and propylene glycol (e.g., pluronics, PF68). The amount of surfactant added is such that it maintains aggregation of the reconstituted protein at an acceptable level as assayed using, e.g., SEC-HPLC to determine the percentage of HMW species or LMW species, and minimizes the formation of particulates after reconstitution of a lyophilate of a protein formulation described herein. For example, the surfactant can be present in a formulation (liquid, or prior to reconstitution of a lyophilate) in an amount from about 0.001 to about 0.5%, e.g., from about 0.05 to about 0.3%.

In some embodiments, a bulking agent is included in the formulation. The term “bulking agent” as used herein, includes agents that provide the structure of the freeze-dried product without interacting directly with the pharmaceutical product. In addition to providing a pharmaceutically elegant cake, bulking agents may also impart useful qualities in regard to modifying the collapse temperature, providing freeze-thaw protection, and enhancing the protein stability over long-term storage. Non-limiting examples of bulking agents include mannitol, glycine, lactose, and sucrose. Bulking agents may be crystalline (such as glycine, mannitol, or sodium chloride) or amorphous (such as dextran, hydroxyethyl starch) and are generally used in protein formulations in an amount from 0.5% to 10%.

Other pharmaceutically acceptable carriers, excipients, or stabilizers, such as those described in Remington's Pharmaceutical Sciences 16th edition, Osol, A. Ed. (1980) may also be included in a protein formulation described herein, provided that they do not adversely affect the desired characteristics of the formulation. As used herein, “pharmaceutically acceptable carrier” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, α-monothioglycerol, and sodium thio sulfate; low molecular weight proteins, such as human serum albumin, bovine serum albumin, gelatin, or other immunoglobulins; and hydrophilic polymers, such as polyvinylpyrrolidone.

Storage Methods

A protein formulation described herein may be stored by any method known to one of skill in the art. Non-limiting examples include freezing, lyophilizing, and spray drying the protein formulation.

In some cases, the protein formulations are frozen for storage. Accordingly, it is desirable that the formulation be relatively stable under such conditions, including under freeze-thaw cycles. One method of determining the suitability of a formulation is to subject a sample formulation to at least two, e.g., three to ten cycles of freezing (at, for example −20° C. or −80° C.) and thawing (for example by fast thaw at room temperature or slow thaw on ice), determining the amount of low molecular weight (LMW) species and/or HNMW species that accumulate after the freeze-thaw cycles and comparing it to the amount of LMW species or HMW species present in the sample prior to the freeze-thaw procedure. An increase in the LMW or HMW species indicates decreased stability of a protein stored as part of the formulation. Size exclusion high performance liquid chromatography (SEC-HPLC) can be used to determine the presence of LMW and HMW species.

In some cases, the protein formulations may be stored as a liquid. Accordingly, it is desirable that the liquid formulation be relatively stable under such conditions, including at various temperatures. One method of determining the suitability of a formulation is to store the sample formulation at several temperatures (such as 2-8, 15, 20, 25, 30, 35, 40, and 50° C.) and monitoring the amount of HMW and/or LMW species that accumulate over time. The smaller the amounts of HMW and/or LMW species that accumulate over time, the better the storage condition for the formulation. Additionally, the charge profile of the protein may be monitored by cation exchange-high performance liquid chromatography (CEX-HPLC).

Alternatively, formulations can be stored after lyophilization. The term “lyophilization” as used herein, refers to a process by which the material to be dried is first frozen followed by removal of the ice or frozen solvent by sublimation in a vacuum environment. An excipient (e.g., lyoprotectant) may be included in formulations that are to be lyophilized so as to enhance stability of the lyophilized product upon storage. The term “reconstituted formulation” as used herein, refers to a formulation that has been prepared by dissolving a lyophilized protein formulation in a diluent such that the protein is dispersed in the diluent. The term “diluent” as used herein, is a substance that is pharmaceutically acceptable (safe and non-toxic for administration to a human) and is useful for the preparation of a liquid formulation, such as a formulation reconstituted after lyophilization. Non-limiting examples of diluents include sterile water, bacteriostatic water for injection (BWFI), a pH buffered solution (e.g., phosphate-buffered saline), sterile saline solution, Ringer's solution, dextrose solution, or aqueous solutions of salts and/or buffers.

Testing a formulation for the stability of the protein component of the formulation after lyophilization is useful for determining the suitability of a formulation. The method is similar to that described above for freezing, except that the sample formulation is lyophilized instead of frozen, reconstituted using a diluent, and the reconstituted formulation is tested for the presence of LMW species and/or HMW species. An increase in LMW or HMW species in the lyophilized sample compared to a corresponding sample formulation that was not lyophilized indicates decreased stability in the lyophilized sample.

In some cases, a formulation is spray-dried and then stored. For spray-drying, a liquid formulation is aerosolized in the presence of a dry gas stream. Water is removed from the formulation droplets into the gas stream, resulting in dried particles of the drug formulation. Excipients may be included in the formulation to (i) protect the protein during the spray-drying dehydration, (ii) protect the protein during storage after spray-drying, and/or (iii) give the solution properties suitable for aerosolization. The method is similar to that described above for freezing, except that the sample formulation is spray-dried instead of frozen, reconstituted in a diluent, and the reconstituted formulation is tested for the presence of LMW species and/or HMW species. An increase in LMW or HMW species in the spray-dried sample compared to a corresponding sample formulation that was not lyophilized indicates decreased stability in the spray-dried sample.

Methods of Treatment

The formulations described herein are useful as pharmaceutical compositions in the treatment and/or prevention of a disease or disorder in a patient in need thereof. The term “treatment” refers to both therapeutic treatment and prophylactic or preventative measures. Treatment includes the application or administration of the protein formulation to the body, an isolated tissue, or cell from a patient who has a disease/disorder, a symptom of a disease/disorder, or a predisposition toward a disease/disorder, with the purpose to cure, heal, alleviate, relieve, alter, remedy, ameliorate, improve, or affect the disease, the symptom of the disease, or the predisposition toward the disease. Those “in need of treatment” include those already with the disorder, as well as those in which the disorder is to be prevented. The term “disorder” is any condition that would benefit from treatment with the protein formulation described herein. This includes chronic and acute disorders or diseases including those pathological conditions that predispose the mammal to the disorder in question. Non-limiting examples of disorders to be treated herein include, bleeding disorders, thrombosis, leukemia, lymphoma, non-Hodgkin's lymphoma, autoimmune disorders, coagulation disorders, hemophilia, graft rejection, inflammatory disorders, heart disease, muscle wasting disorders, allergies, cancers, muscular dystrophy, sarcopenia, cachexia, Type II diabetes, rheumatoid arthritis, Crohn's disease, psoriasis, psoriatic arthritis, asthma, dermatitis, allergic rhinitis, chronic obstructive pulmonary disease, eosinophilia, fibrosis, and excess mucus production.

Administration

The protein formulations described herein can be administered to a subject in need of treatment using methods known in the art, such as by single or multiple bolus or infusion over a long period of time in a suitable manner, e.g., injection or infusion by subcutaneous, intravenous, intraperitoneal, intramuscular, intraarterial, intralesional or intraarticular routes, topical administration, transmucosal, transdermal, rectal, inhalation, or by sustained release or extended-release means. If the protein formulation has been lyophilized, the lyophilized material is first reconstituted in an appropriate liquid prior to administration. The lyophilized material may be reconstituted in, e.g., bacteriostatic water for injection (BWFI), physiological saline, phosphate buffered saline (PBS), or the same formulation the protein had been in prior to lyophilization.

Parenteral compositions can be prepared in dosage unit form for ease of administration and uniformity of dosage. “Dosage unit form” as used herein, refers to physically discrete units suited as unitary dosages for the subject to be treated; each unit containing a predetermined quantity of active compound calculated to produce the desired therapeutic effect in association with the selected pharmaceutical carrier.

In the case of an inhalation method, such as metered dose inhaler, the device is designed to deliver an appropriate amount of the formulation. For administration by inhalation, the compounds are delivered in the form of an aerosol spray from a pressured container or dispenser that contains a suitable propellant, e.g., a gas, such as carbon dioxide, or a nebulizer. Alternatively, an inhaled dosage form may be provided as a dry powder using a dry powder inhaler.

The protein formulation may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles, and nanocapsules) or in macroemulsions. Such techniques are disclosed in Remington's Pharmaceutical Sciences 18th edition.

Sustained-release preparations of the protein formulations described herein may also be prepared. Suitable examples of sustained-release preparations include semipermeable matrices of solid hydrophobic polymers containing the protein formulation. Examples of sustained-release matrices include polyesters, hydrogels (for example, poly(2-hydroxyethyl-methacrylate), or poly(vinylalcohol)), polylactides, copolymers of L-glutamic acid and γ-ethyl-L-glutamate, non-degradable ethylene-vinyl acetate, degradable lactic acid-glycolic acid copolymers, and poly-D-(−)-3-hydroxybutyric acid. The sustained-release formulations of the proteins described herein may be developed using polylactic-coglycolic acid (PLGA) polymer due to its biocompatibility and wide range of biodegradable properties. The degradation products of PLGA, lactic and glycolic acids, can be cleared quickly within the human body. Moreover, the degradability of this polymer can be adjusted from months to years depending on its molecular weight and composition. Liposomal compositions may also be used to formulate the proteins or antibodies disclosed herein.

Dosing

Toxicity and therapeutic efficacy of a formulation can be determined by pharmaceutical procedures known in the art using, e.g., cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀.

The data obtained from the cell culture assays and animal studies can be used in formulating a range of dosage for use in humans. The dosage of such formulations generally lies within a range of circulating concentrations that include the ED₅₀ with little or no toxicity. The dosage may vary within this range depending upon the dosage form employed and the route of administration utilized. For any formulation used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

The appropriate dosage of the protein of the formulation will depend on the type of disease to be treated, the severity and course of the disease, whether the agent is administered for preventive or therapeutic purposes, previous therapy, the patient's clinical history and response to the agent, and the discretion of the attending physician. A formulation is generally delivered such that the dosage is between about 0.1 mg protein/kg of body weight to 100 mg protein/kg of body weight.

In order for the formulations to be used for in vivo administration, they must be sterile. The formulation may be rendered sterile by filtration through sterile filtration membranes, prior to, or following, formulation of a liquid or lyophilization and reconstitution. The therapeutic compositions herein generally are placed into a container having a sterile access port, for example, an intravenous solution bag, or vial having a stopper pierceable by a hypodermic injection needle.

Articles of Manufacture

In another embodiment, an article of manufacture is provided which contains a formulation described herein and preferably provides instructions for its use. The article of manufacture comprises a container suitable for containing the formulation. Suitable containers include, without limitation, bottles, vials (e.g., dual chamber vials), syringes (e.g., single or dual chamber syringes), test tubes, nebulizers, inhalers (e.g., metered dose inhalers or dry powder inhalers), or depots. The container can be formed from a variety of materials, such as glass, metal or plastic (e.g., polycarbonate, polystyrene, polypropylene). The container holds the formulation and the label on, or associated with, the container may indicate directions for reconstitution and/or use. The label may further indicate that the formulation is useful or intended for subcutaneous administration. The container holding the formulation may be a multi-use vial, which allows for repeat administrations (e.g., from 2-6 administrations) of the formulation. The article of manufacture may further comprise a second container comprising a suitable diluent (e.g., WFI, 0.9% NaCl, BWFI, phosphate buffered saline). When the article of manufacture comprises a lyophilized version of a protein formulation, mixing of a diluent with the lyophilized formulation will provide a final protein concentration in the reconstituted formulation of generally at least 20 mg/ml. The article of manufacture may further include other materials desirable from a commercial and user standpoint, including other buffers, diluents, filters, needles, syringes, and package inserts with instructions for use.

All journal articles, patents, patent applications, and other publications referenced in this application are incorporated by reference in their entirety. If there is any conflict between the contents of the instant application and any of the material incorporated by reference, it is to be understood that this application governs.

The invention will be more fully understood by reference to the following examples. They should not, however, be construed as limiting the scope of the invention.

### EXAMPLES

### Example 1

### Effect of Methionine on Protein Aggregation in an anti-B7.2 Antibody Formulation Subjected to Storage at Elevated Temperature

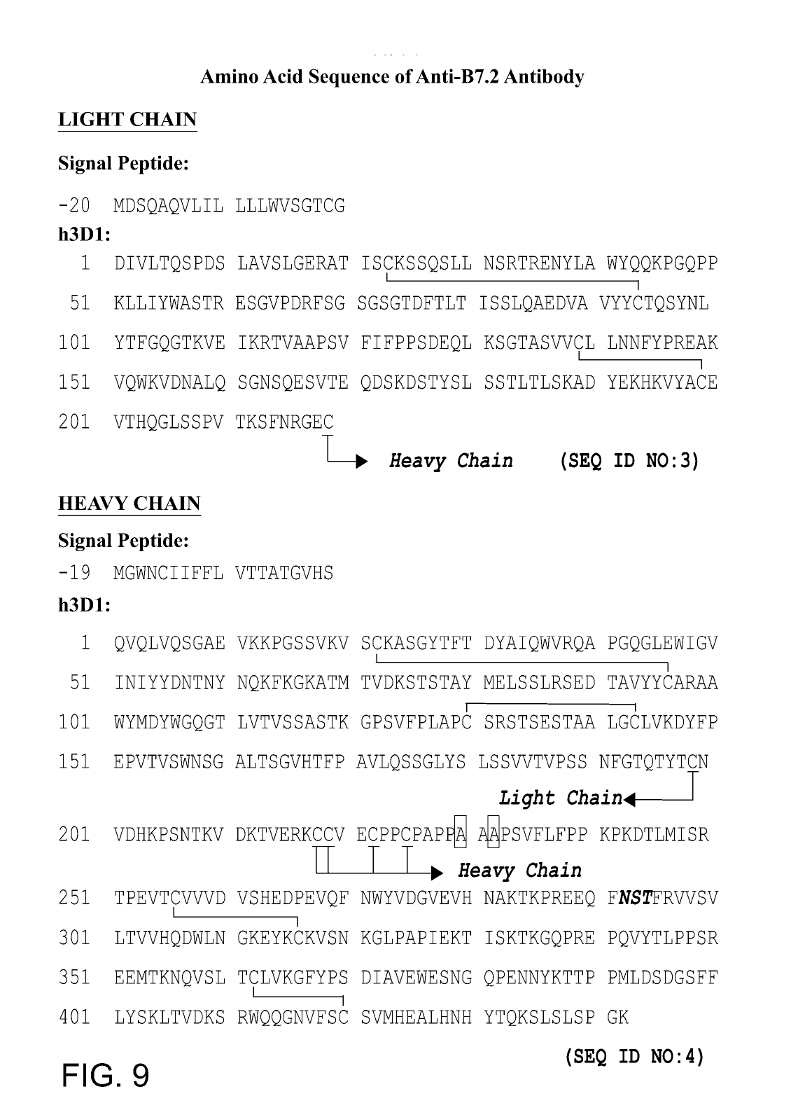

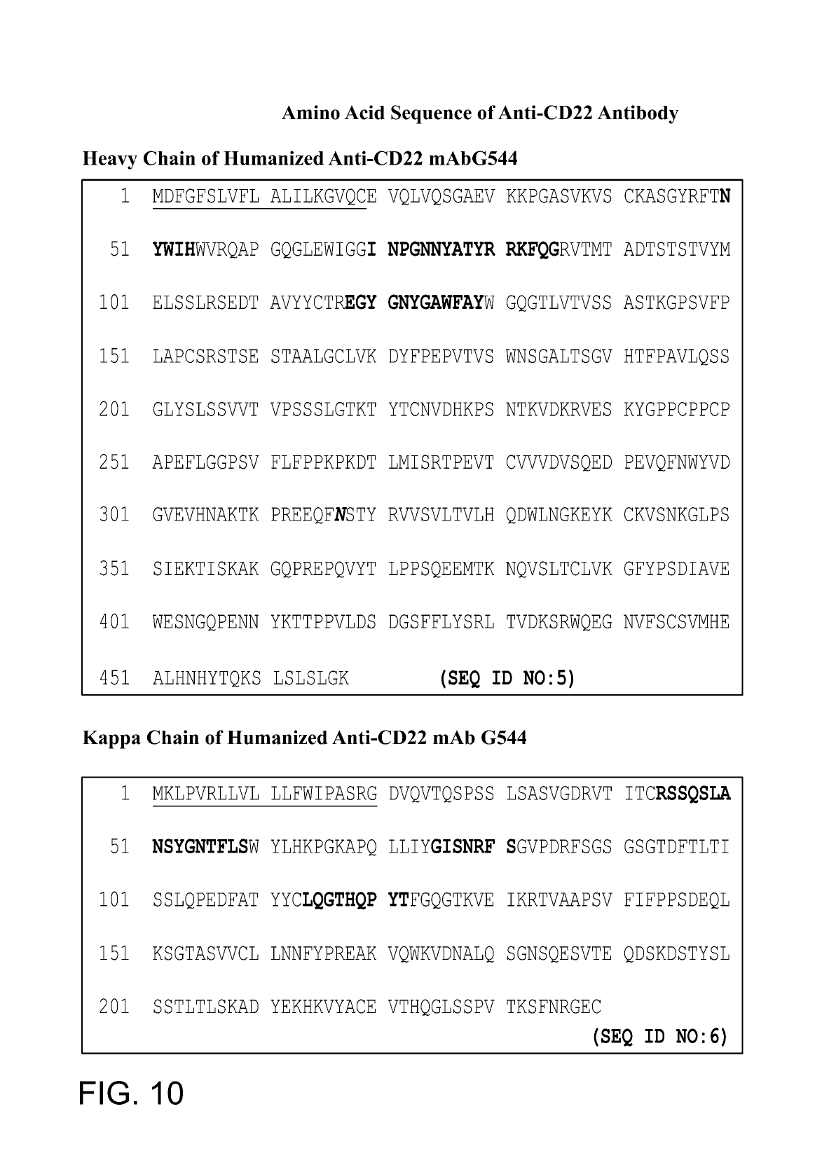

This example illustrates the ability of methionine to reduce aggregation of a protein in a protein formulation. Specifically, the experiments described below were directed at testing the effects of methionine on the aggregation of anti-B7.2 antibodies (IgG₂, κ light chain, see, FIG. 9) in an anti-B7.2 antibody formulation subjected to storage at 40° C. B7.2 is a co-stimulatory ligand that is expressed on B cells, which can interact with the T cell surface molecules, CD28 and CTLA-4.

The effect of adding methionine on the aggregation of anti-B7.2 antibody formulated as a liquid at various pH levels was examined over a 12-week period during which the formulation was stored at 40° C. The anti-B7.2 antibody was formulated at 1 mg/ml at various pH levels in the presence and absence of 10 mM methionine and 0.01% polysorbate-80. Aggregation levels were measured initially, at week 6, and at week 12, by measuring the percentage of high molecular weight (% HMW) species in the formulations at these time points by SEC-HPLC. An increase in % HMW is indicative of aggregation.

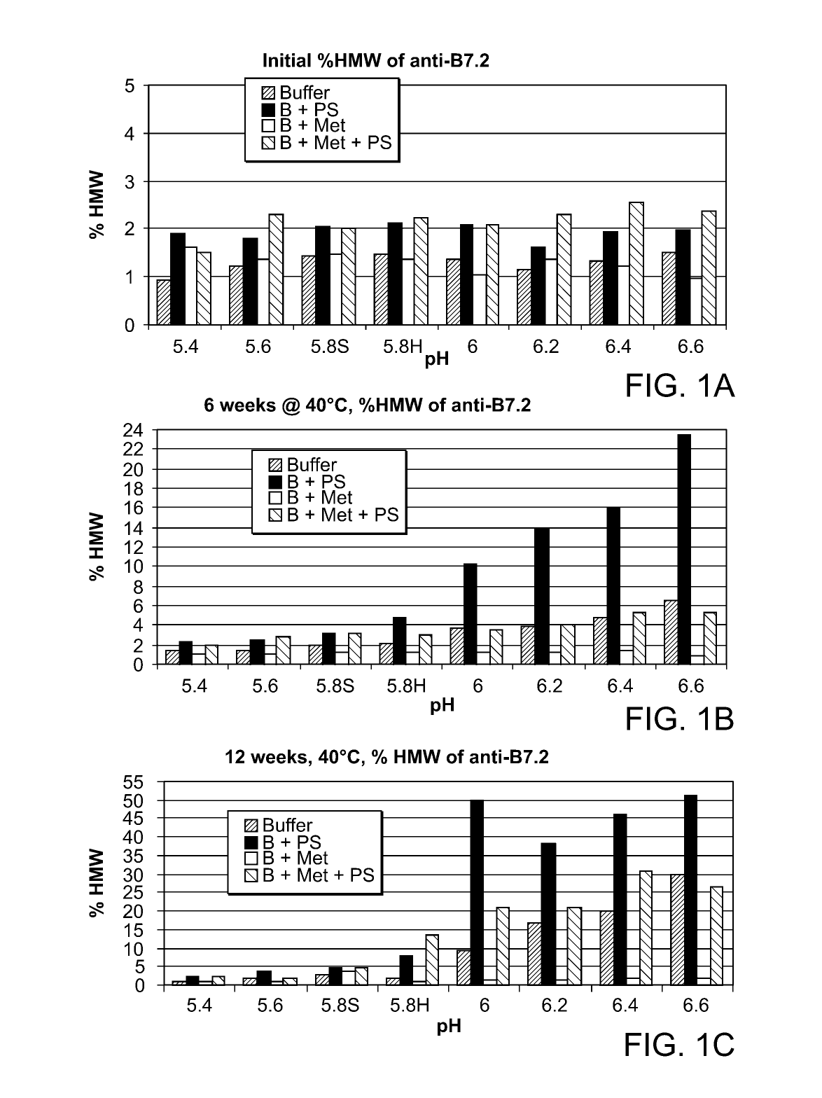

Initial % HMW levels of each formulation were approximately the same (˜1-2% see, FIG. 1a). After 6 and 12 weeks of storage at 40° C., however, % HMW increased in formulations lacking methionine, especially in the samples containing polysorbate-80 and lacking methionine and formulated at pH levels ranging from 6.0 to 6.6 (see, FIGS. 1b and 1c). The presence of methionine in the formulation kept % HMW near initial levels in samples without polysorbate-80. Although there was an increase in protein aggregation in samples containing both polysorbate-80 and methionine compared with samples containing methionine but lacking polysorbate-80, the levels of protein aggregation were significantly lower than in the samples containing polysorbate-80 but lacking methionine (see, FIGS. 1b and 1c).

In summary, these experiments show that methionine reduced aggregation of anti-B7.2 antibody in a formulation subjected to elevated temperatures both in the presence and absence of polysorbate-80.

### Example 2

### Effect of Methionine on Protein Aggregation in an Anti-B7.1 Antibody Formulation Subjected to Elevated Temperature

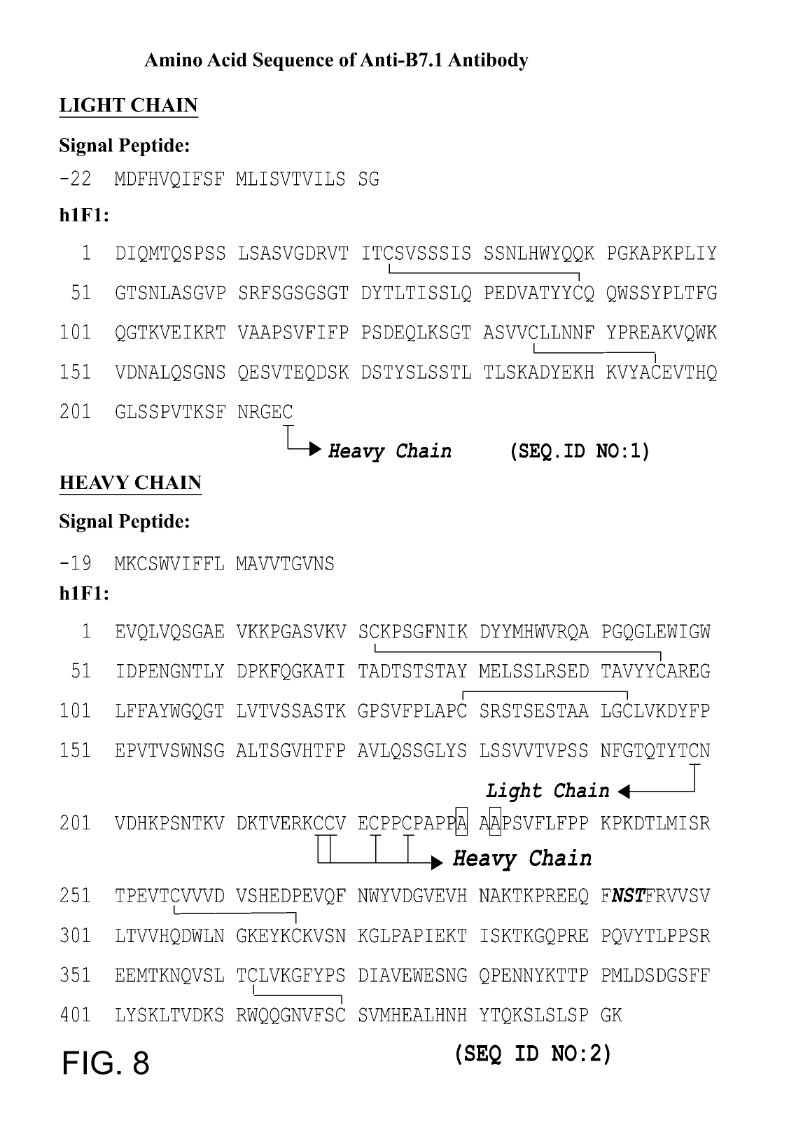

This example further illustrates the ability of methionine to reduce aggregation of a protein in a protein formulation. The experiments described below were directed at testing the effects of methionine on the aggregation of anti-B7.1 antibodies (IgG₂, κ light chain, see, FIG. 8) in an anti-B7.1 antibody formulation subjected to storage at 40° C. B7.1 is a co-stimulatory ligand that is expressed on B cells, which can interact with the T cell surface molecules, CD28 and CTLA-4.

The effect of adding methionine on the aggregation of anti-B7.1 antibody formulated as a liquid at various pH levels was examined over a 12-week period during which the samples were stored at 40° C. The anti-B7.1 antibody was formulated at 1 mg/ml at various pH levels in the presence and absence of 10 mM methionine and 0.01% polysorbate-80. Aggregation levels were measured initially, and at week 12, by measuring the percentage of high molecular weight (% HMW) species in the formulations at these time points by SEC-HPLC.

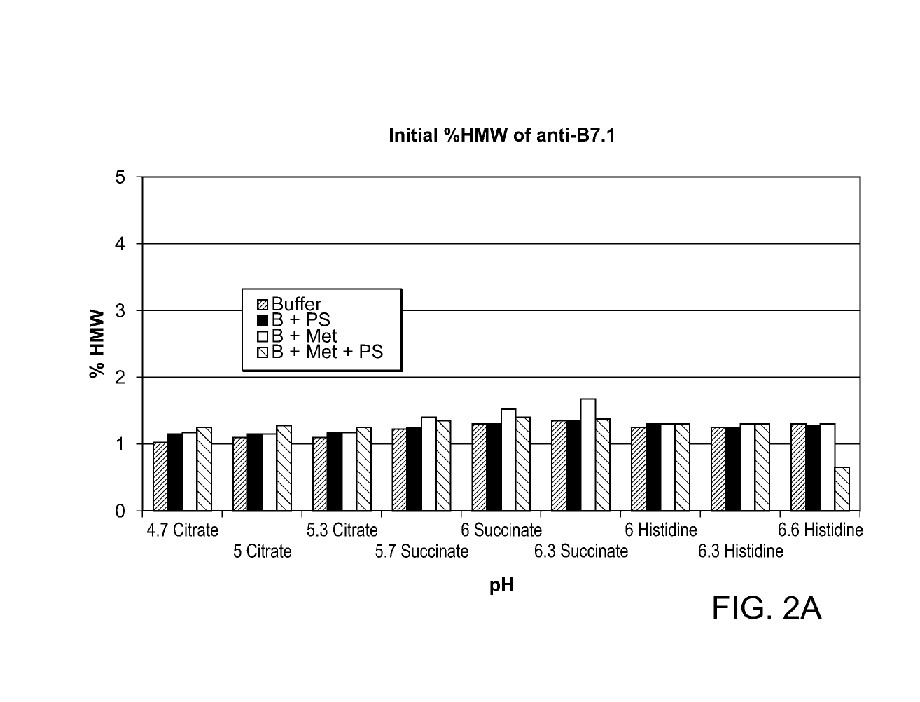

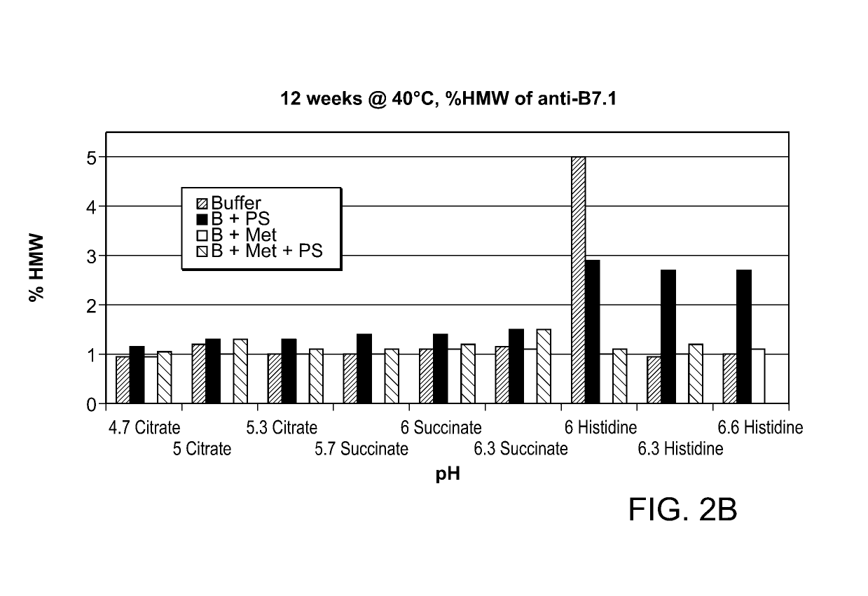

Initial % HMW levels of each formulation was approximately the same (˜1%, see, FIG. 2a). Storage of the anti-B7.1 antibody formulation for 12 weeks at 40° C. in the presence of polysorbate-80 and lacking methionine resulted in a minor increase in % HMW in the pH range of 4.7-6.3 in citrate and succinate buffers (see, FIG. 2b). A more significant increase in % HMW resulted in the pH range of 6-6.6 in hisfidine buffer (see, FIG. 2b). The addition of methionine to the protein formulations decreased % HMW levels. This was most clearly seen in the case of anti-B7.1 antibody formulated in histidine buffer and polysorbate-80: methionine kept the % HMW levels to a minimal 1.2% after 12 weeks at 40° C.

In summary, these experiments show that methionine reduced aggregation of anti-B7.1 antibody in a formulation stored at 40° C., both in the presence and absence of polysorbate-80.

### Example 3

### Effect of Methionine on Protein Aggregation in an Anti-CD22 Antibody Formulation Subjected to Long-Term Storage

This experiment was directed to testing the effect of adding methionine on protein aggregation in an anti-CD22 antibody formulation (see, FIG. 10). CD22 is a 135 kD B-cell restricted sialoglycoprotein that binds to oligosaccharides containing 2-6-linked sialic acid residues, and is expressed on the surface of B-cells during later stages of differentiation. It appears to play a role in B-cell activation and to act as an adhesion molecule. CD22 and anti-CD22 are considered useful in the treatment of leukemia, lymphoma, non-Hodgkin's lymphoma, and certain autoimmune conditions.

25-26 mg/ml of anti-CD22 (IgG₄, κ light chain) was formulated as a liquid in 10 mM succinate buffer, pH 6. These formulations also contained either one or both of 10 mM methionine and 0.01% polysorbate-80. The resulting anti-CD22 formulations were stored at 25° C. or −80° C. for between 1 month to 36 months, and the % HMW levels in the formulations was assessed by SEC-HPLC.

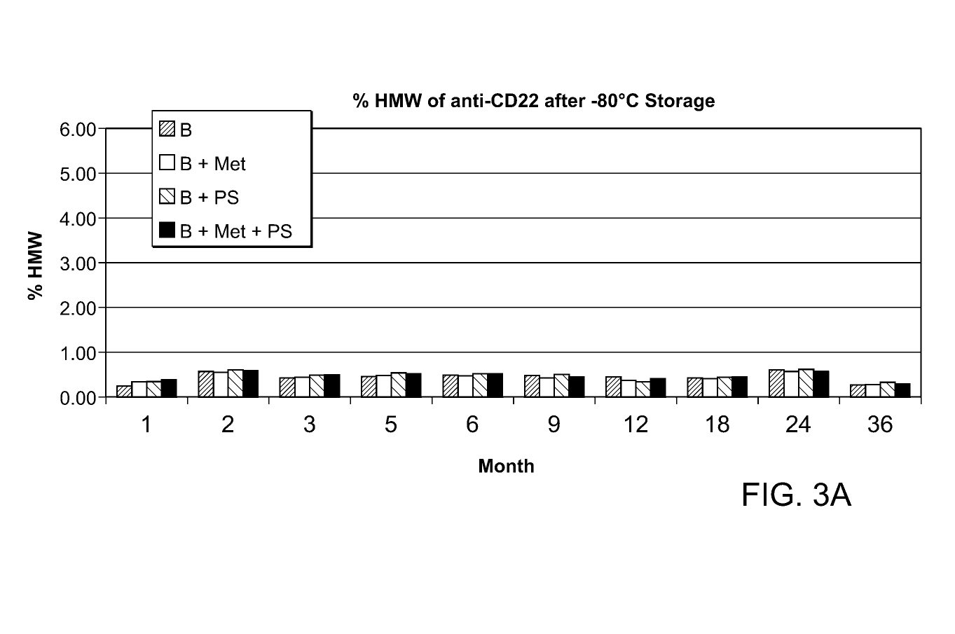

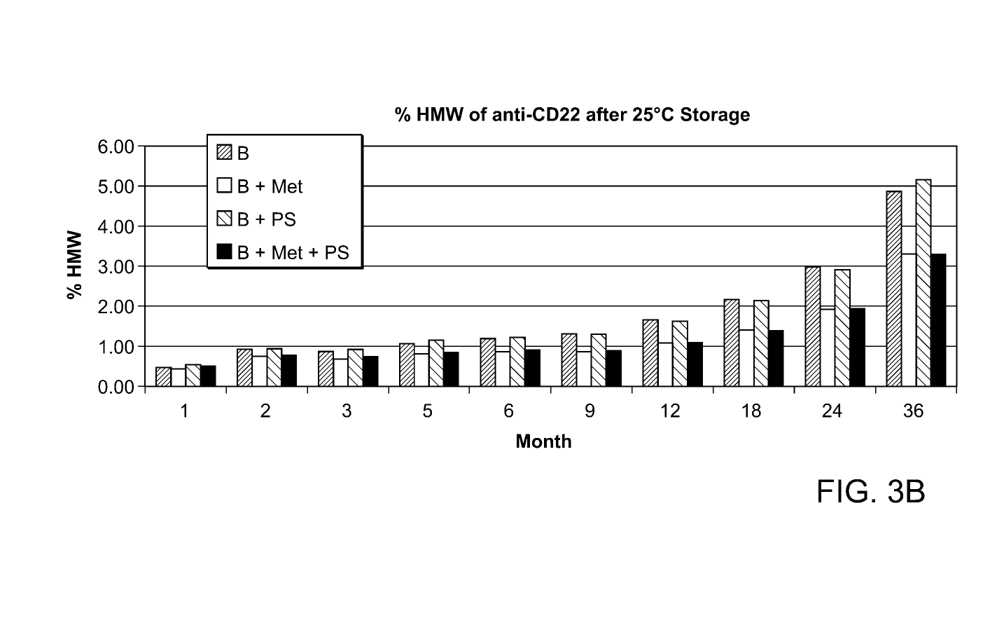

The % HMW levels of all formulations stored at −80° C. were approximately the same (˜0.5%) (see, FIG. 3a). In contrast, storage over time at 25° C. resulted in an increase in the % HMW levels (see, FIG. 3b). This increase was substantially decreased if methionine was present in the formulation. Of note, anti-CD22 formulations formulated with polysorbate-80 and methionine generated approximately the same % HMW species as samples formulated with methionine but lacking polysorbate-80.

These data indicate that methionine decreases protein aggregation of an anti-CD22 antibody formulation in long-term storage, both in the presence or absence of polysorbate-80.

### Example 4

### Effect of Methionine on Protein Aggregation in a PSGL-Ig Formulation Subjected to Storage at High Temperatures

This example provides another illustration of methionine's ability to prevent aggregation in proteins and, particularly, in fusion proteins. This experiment was directed to testing the effect of adding methionine on protein aggregation in a P-selectin glycoprotein ligand-1-immuoglobulin (PSGL-Ig) fusion protein formulation. PSGL-1 is a 240 kDa homodimer consisting of two 120 kDa polypeptide chains that is constitutively expressed on all leukocytes. PSGL-1 is primarily found on the tips of the microvilli. PSGL-1 can bind to P-selectin on the endothelium when decorated with appropriate sugars.

The effects of methionine on the aggregation of fusion protein P-selectin glycoprotein ligand-Ig (PSGL-Ig) were examined at various temperatures. PSGL-Ig was formulated as a liquid formulation in 10 mM Tris, 150 mM NaCl, 0.005% polysorbate-80, pH 7.5 in the presence and absence of 10 mM methionine. Samples were stored at −80° C., 25° C., and 40° C. and were evaluated for % HMW over a 4-week period by SEC-HPLC.

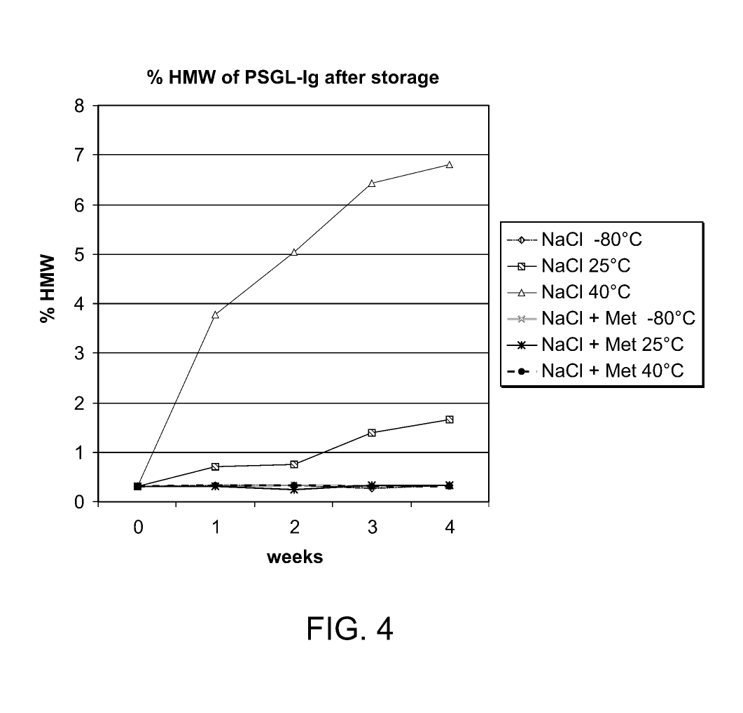

Initial % HMW levels in all samples were similar and remained unchanged in the samples stored at −80° C. regardless of the presence or absence of methionine (see, FIG. 4). Storage at 25° C. and 40° C. resulted in increased aggregation over time; however, that aggregation was reduced in samples formulated with methionine.

### Example 5

### Effect of Methionine on Protein Aggregation in a PSGL-Ig Formulation Subjected to Shear Stress

This example illustrates that methionine reduces aggregation of proteins subjected to shear stress.

PSGL-Ig fusion protein was formulated as a liquid in 10 mM Tris, 150 mM NaCl, 0.005% polysorbate-80, pH 7.5 in the presence and absence of 10 mM methionine. The resulting formulations were either left unshaken or subjected to shaking at 250 rpm for 96 hours.

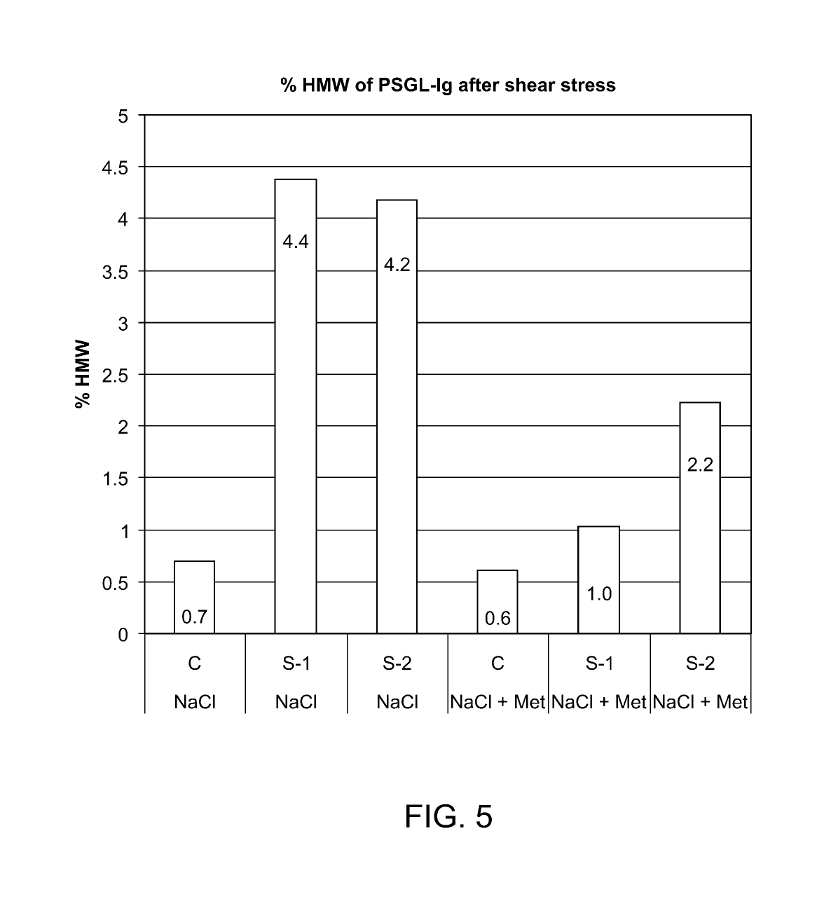

Unshaken samples containing or lacking methionine had very similar % HMW levels (0.6 and 0.7%) (see, FIG. 5). In contrast, shaken samples lacking methionine contained elevated % HMW (4.2% and 4.4%). Addition of methionine to formulations that were subjected to shaking resulted in a decrease in the % HMW levels to 1.0 and 2.2%.

These data show that methionine reduces aggregation of proteins subjected to shear stress.

### Example 6

### Effect of Methionine on Protein Aggregation of a REFACTO® Protein Formulation Stored in the Dark

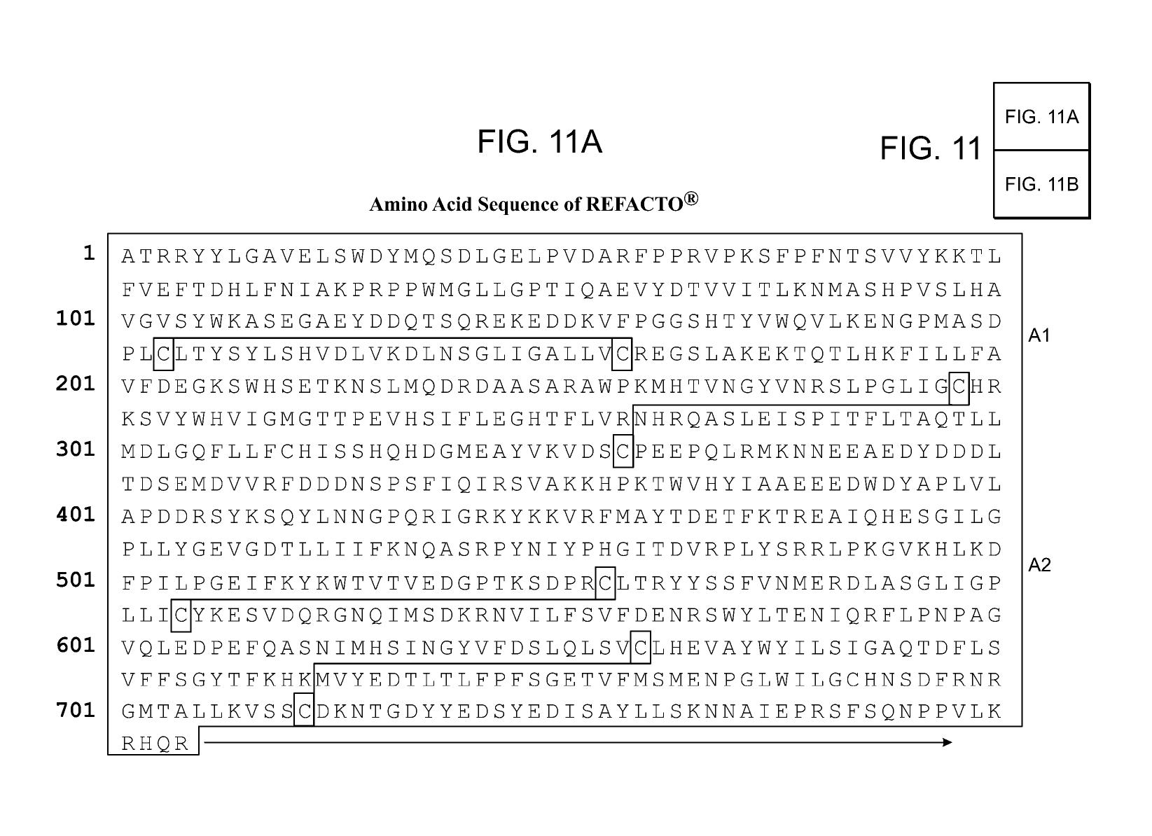

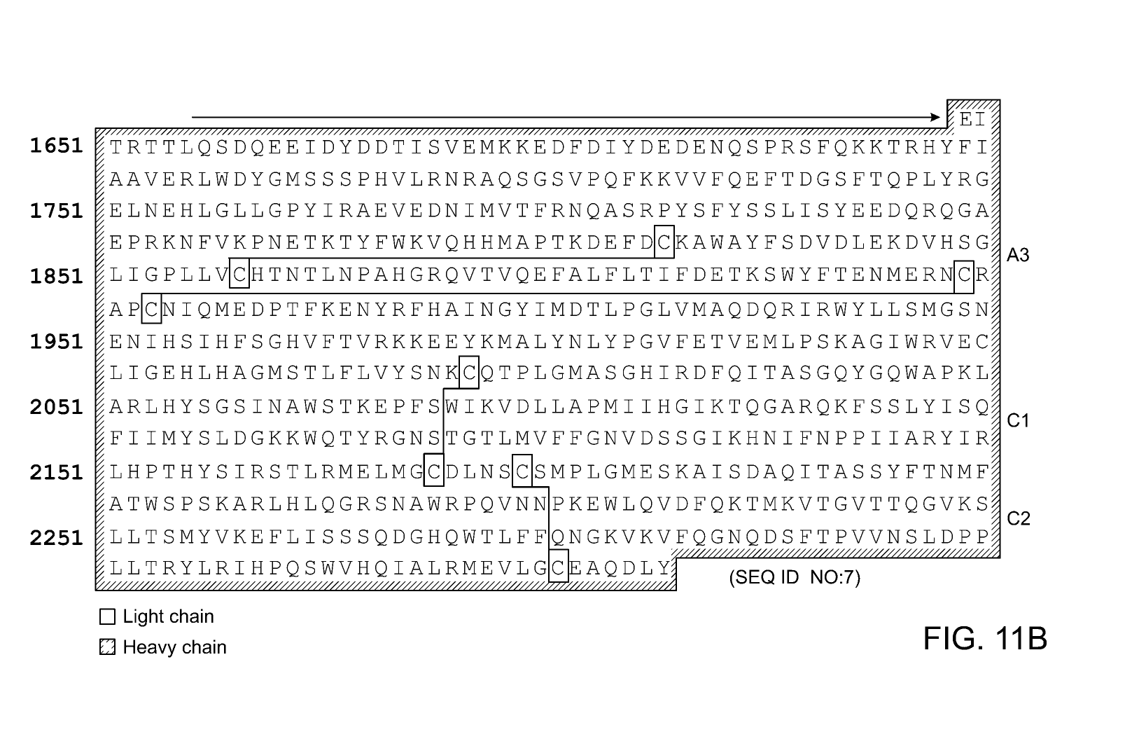

This experiment provides yet another example of methionine's ability to prevent aggregation in proteins and, particularly, in recombinant proteins. To further illustrate, REFACTO® (see, FIG. 11), a recombinant factor VIII protein that is used to correct factor VIII deficiencies, was used in this experiment.

The effects of methionine on the stability of REFACTO® were examined over a 1-month stability study. REFACTO® was formulated as a liquid at about 250 IU/ml in 20 mM histidine buffer. Some of these formulations also contained 10 mM methionine and 10 mM citrate. All of the formulations contained 4 mM calcium chloride and 310 mM sodium chloride, and 0.02% Tween-80. The pH of the formulations was 6.5. Samples were stored in the dark at room temperature for approximately 1 month. Control samples were formulated as above and stored at −80° C. Aggregate formation was assessed by SEC-HPLC.

In control samples, regardless of the presence of methionine and citrate, % HMW levels remained the same (see, Table 1). In histidine buffer formulations without methionine and citrate, % HMW was 26-27% after 1 month of storage in the dark, indicating a high level of aggregation. In histidine buffer formulations containing methionine and citrate, however, aggregation was reduced over the same time period, with a % HMW of only 7-8%.

TABLE 1

| | | [Methionine + | % |
|---|---|---|---|
| Storage | [Buffer] | Citrate] | HMW |
| Control | 20 mM reagent | none | 1.1 |
| (Stored at −80° C.) | grade Histidine | | |
| Dark | 20 mM reagent | none | 26.6 |
| | grade Histidine | | |
| Control | 20 mM reagent | 10 mM Methionine + | 0.9 |
| (stored at −80° C.) | grade Histidine | 10 mM Citrate | |
| Dark | 20 mM reagent | 10 mM Methionine + | 7.9 |
| | grade Histidine | 10 mM Citrate | |
| Control | 20 mM USP | none | 0.0 |
| (stored at −80° C.) | grade Histidine | | |
| Dark | 20 mM USP | none | 25.8 |
| | grade Histidine | | |
| Control | 20 mM USP | 10 mM Methionine + | 0.0 |
| (stored at −80° C.) | grade Histidine | 10 mM Citrate | |
| Dark | 20 mM USP | 10 mM Methionine + | 7.7 |
| | grade Histidine | 10 mM Citrate | |

### Example 7

### Effect of Methionine on Protein Aggregation of a REFACTO® Protein Formulation Stored Under Fluorescent Light

In this set of experiments, the effects of methionine on fragmentation of REFACTO® that was exposed to fluorescent light were examined over a 1-month period.

REFACTO® was formulated as a liquid at about 250 IU/ml in 20 mM histidine or 20 mM succinate buffer. Some of these formulations also contained 10 mM methionine and 10 mM citrate. All of the formulations contained 4 mM calcium chloride and 310 mM sodium chloride, and 0.02% Tween-80. The pH of the formulations was 6.5. Samples were stored at room temperature for approximately 1 month under fluorescent light, and aggregate formation was assessed by SEC-HPLC. Control samples were formulated as above and stored at −80° C.

In control samples, regardless of the presence of methionine and citrate, % HMW levels remained unchanged at 0% HMW (see, Table 2). In USP grade histidine buffered formulations without methionine and citrate, % HMW was 21% after 1 month of storage under fluorescent light, indicating a high level of aggregation. In USP grade histidine buffered formulations containing methionine and citrate, however, aggregation was reduced over the same time period, with a % HMW of only about 2%.

Similarly, in succinate buffered formulations lacking methionine and citrate, % HMW was 25%, whereas succinate buffered formulations containing methionine and citrate had only 9% HMW (see, Table 3).

Thus, methionine and citrate decreased aggregation of REFACTO® formulated in histidine or succinate buffers and stored under fluorescent light, compared with REFACTO® formulated without methionine and citrate.

TABLE 2

| | | [Methionine + | % |
|---|---|---|---|
| Storage | [Buffer] | Citrate] | HMW |
| Control | 20 mM USP | none | 0.0 |
| (stored at −80° C.) | grade Histidine | | |
| Light | 20 mM USP | none | 21.2 |
| | grade Histidine | | |
| Control | 20 mM USP | 10 mM Methionine + | 0.0 |
| (stored at −80° C.) | grade Histidine | 10 mM citrate | |
| Light | 20 mM USP | 10 mM Methionine + | 1.7 |
| | grade Histidine | 10 mM citrate | |

TABLE 3

| | | [Methionine + | % |
|---|---|---|---|
| Storage | [Buffer] | Citrate] | HMW |
| Control | 20 mM reagent | none | 1.1 |
| (Stored at −80° C.) | grade succinate | | |
| Light | 20 mM reagent | none | 24.7 |
| | grade succinate | | |
| Control | 20 mM reagent | 10 mM Methionine + | 1.0 |
| (stored at −80° C.) | grade succinate | 10 mM citrate | |
| Light | 20 mM reagent | 10 mM Methionine + | 9.2 |
| | grade succinate | 10 mM citrate | |

### Example 8

### Effect of Methionine on Potency of REFACTO®

The effects of methionine on the potency of REFACTO® that was either kept in the dark or exposed to fluorescent light were examined over a 1-month period. REFACTO® was formulated as a liquid at about 250 IU/ml in 20 mM histidine or 20 mM succinate buffer. Some of these formulations also contained 10 mM methionine and 10 mM citrate. Samples were exposed to fluorescent light or dark conditions for 1 month at room temperature.

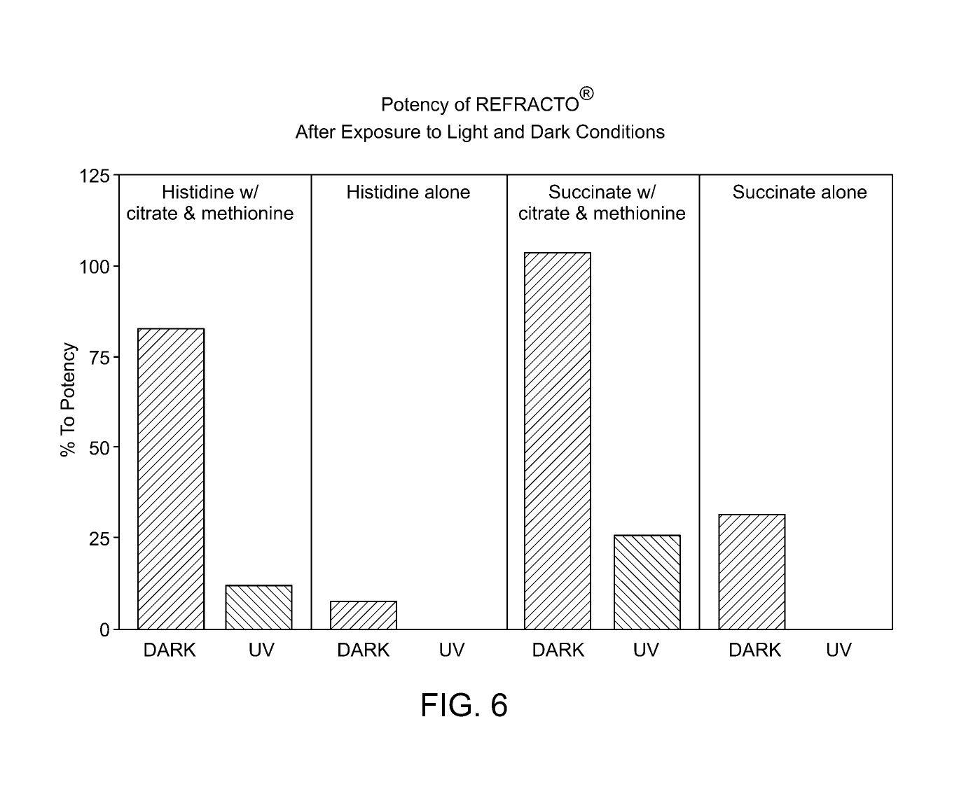

REFACTO® suffered a large loss of potency in the buffered solutions formulated without methionine and citrate after 1 month of storage at room temperature in either samples exposed to fluorescent light (see, FIG. 6). REFACTO® stored in the dark in the presence of methionine suffered no deleterious effects on potency, whereas REFACTO® stored under fluorescent light in the presence of methionine suffered some loss in potency but still retained a higher potency than samples stored without methionine, which resulted in a complete loss of potency.

### Example 9

### Oxidation Decreases Multimerization of rhIL-11

This experiment was directed at testing the effect of methionine addition on IL-11 multimerization.

Four hundred vials were hand filled at 0.1 mg/ml with recombinant human IL-11 (rhIL-11) drug substance (1.0 ml fill in a 5 ml tubing vial) and lyophilized using a standard lyophilization cycle for rhIL-11. Two hundred vials contained rhIL-11 formulated with 10 mM NaPO₄, 300 mM glycine, pH 7.0, and the remainder were formulated with 10 mM NaPO₄, 300 mM glycine, 10 mM methionine, pH 7.0. Four different 13 mm stoppers were used as container closures. Each type of stopper was used on 100 vials. The stoppers were rinsed, boiled, and then autoclaved. Half of the stoppers were then dried for 16 hours at 100° C. Vials were placed on short-term accelerated stability at 4° C., 40° C., and 50° C. for two and four weeks. Vials were assayed at T=0 and at 2 and 4 weeks for Met⁵⁸ oxidation and multimer formation. RP-HPLC (low load) was used to determine the degree of oxidation of Met⁵⁸ in rhIL-11, whereas SEC-HPLC was used to monitor the generation of rhIL-11 multimer.

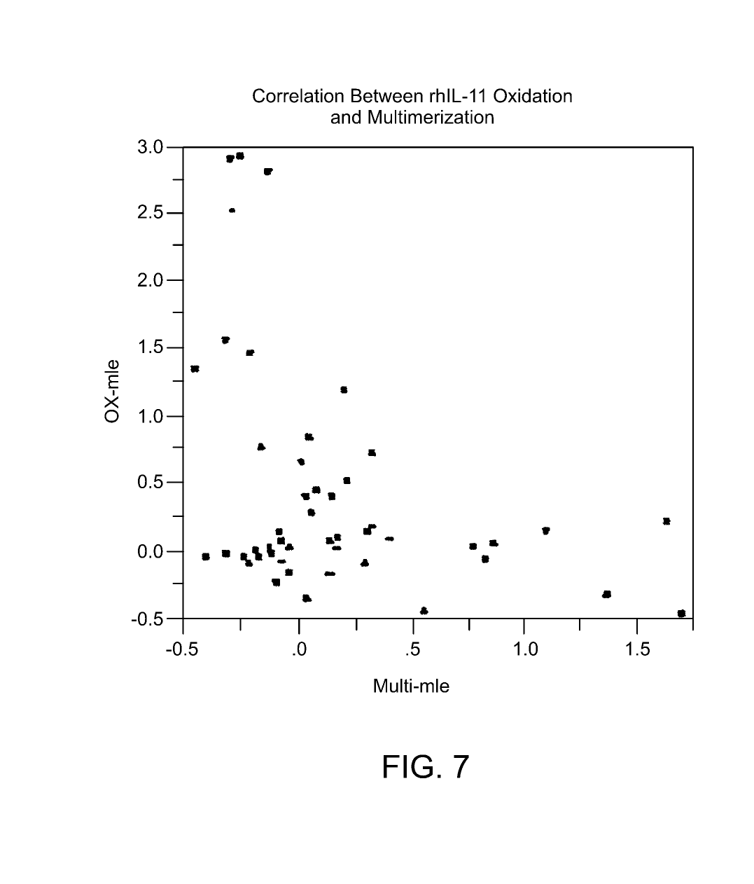

An initial plot was constructed to test for any direct correlation between oxidation and multimerization (see, FIG. 7). These data showed that when levels of oxidation are high, multimer levels are low, and that when levels of oxidation are low, multimer levels are high.

These data indicate that oxidation and multimerization of rhIL-11 appear to occur under opposite circumstances. When the parameters are optimized to minimize oxidation of rhIL-11, multimerization increases.

1. A method for reducing aggregation of a protein in a protein formulation, comprising adding methionine to the formulation to a concentration of about 0.5 mM to about 145 mM, wherein the method results in reduced aggregation of the protein in the formulation compared with the protein in a formulation lacking methionine.

2. The method of claim 1, wherein the protein formulation is a liquid formulation or a freeze dried powder.

3. The method of claim 1, wherein the protein is at a concentration of between about 0.1 mg/ml and about 300 mg/ml.

4. The method of claim 1, wherein the protein formulation comprises a surfactant.

5. The method of claim 1, wherein the protein formulation comprises an amino acid selected from the group consisting of arginine, lysine, aspartic acid, glycine, and glutamic acid.

6. The method of claim 1, wherein the protein formulation comprises a tonicity modifier.

7. The method of claim 1, wherein the protein formulation comprises a sugar.

8. The method of claim 1, wherein the protein formulation further comprises an agent that reduces aggregation of the protein of the formulation.

9. The method of claim 1, wherein protein aggregation is not the result of methionine oxidation.

10. The method of claim 1, wherein aggregation of the protein of the formulation is assessed before and/or after adding methionine to the formulation.

11. The method of claim 10, wherein aggregation is assessed by SEC-HPLC, AUC, light scattering, and UV absorbance.

12. The method of claim 1, wherein the aggregation is assessed by % HMW species, and the % HMW species is reduced by about 30% compared with % HMW species in a formulation lacking methionine.

13. The method of claim 1, wherein aggregation of the protein of the formulation is assessed between 1 week and 12 weeks after adding methionine to the protein formulation or between 1 month and 36 months after addition of methionine to the protein formulation.

14. The method of claim 1, wherein aggregation of the protein of the formulation is assessed after storage of the protein formulation at a temperature between 4° C. and 50° C. for about 1 week to about 12 weeks after formulating the protein formulation with methionine.

15. The method of claim 1, wherein aggregation of the protein of the formulation is assessed after storage of the protein formulation at a temperature between 4° C. and 30° C. for about 1 month to about 36 months after formulating the protein formulation with methionine.

16. The method of claim 1, wherein aggregation of the protein of the formulation is a result of shear stress, storage, storage at elevated temperature, exposure to light, pH, presence of surfactants, and combinations thereof.

17. The method of claim 1, wherein methionine is added to the formulation to a final concentration of between about 1 mM and 25 mM.

18. The method of claim 1, wherein the formulation has a pH of between about 5.0 and 7.0.

19. The method of claim 1, wherein the protein formulation comprises a buffer selected from the group consisting of citrate, succinate, histidine, Tris, and combinations thereof.

20. The method of claim 1, wherein the method increases the shelf life of the formulation, or maintains the potency of the formulation.

21. The method of claim 1, wherein the protein lacks methionine residues or contains less than 5 methionine residues.

22. A method for reducing aggregation of a protein in a protein formulation subjected to shear stress, comprising adding methionine to the formulation to a concentration of about 0.5 mM to about 145 mM, wherein the method results in reduced aggregation of the protein in the formulation compared with the protein in a formulation lacking methionine.

23. The method of claim 22, wherein the shear stress is the result of shaking, drawing into a syringe and purification procedures, and combinations thereof.

24. A method of reducing a loss in potency or biological activity of a protein in a protein formulation after storage of the formulation at room temperature for more than a day, comprising adding methionine to the formulation to a concentration of about 0.5 mM to about 145 mM, thereby reducing the loss in potency or biological activity of the protein in the formulation compared with the protein in a formulation lacking methionine.

25. The method of claim 24, wherein the protein formulation is stored under fluorescent light.

26. The method of claim 24, wherein the protein formulation is stored in the dark for about 1 month.

27. A method for reducing aggregation of a protein in a protein formulation, comprising:
(i) adding methionine to the formulation to a concentration of about 0.5 mM to about 145 mM; and
(ii) determining the % HMW levels of the protein of the formulation by SEC-HPLC;
wherein the method results in reduced aggregation of the protein in the formulation compared with the protein in a formulation lacking methionine.

28. The method of claim 27, wherein the method results in a protein formulation having less than about 5% HMW species as determined by SEC-HPLC.

29. A protein formulation comprising one of an anti-B7.1 antibody, an anti-B7.2 antibody, an anti-CD22 antibody, PSGL-Ig and Factor VIII, or a biologically active fragment thereof, and about 0.5 mM to 50 mM methionine.

30. The formulation of claim 29, further comprising 1-150 mM of an amino acid selected from the group consisting of arginine, lysine, aspartic acid, and glutamic acid.


SEQUENCE LISTING (7 sequences)

SEQ ID NO: 1 - protein, 237 aa, Artificial Sequence
  Description of Artificial Sequence Synthetic light chain construct
MDFHVQIFSFMLISVTVILSSGDIQMTQSPSSLSASVGDRVTITCSVSSSISSSNLHWYQQKPGKAPKPLIYGTSNLASGVPSRFSGSGSGTDYTLTISSLQPEDVATYYCQQWSSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 461 aa, Artificial Sequence
  Description of Artificial Sequence Synthetic heavy construct
MKCSWVIFFLMAVVTGVNSEVQLVQSGAEVKKPGASVKVSCKPSGFNIKDYYMHWVRQAPGQGLEWIGWIDPENGNTLYDPKFQGKATITADTSTSTAYMELSSLRSEDTAVYYCAREGLFFAYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 239 aa, Artificial Sequence
  Description of Artificial Sequence Synthetic light chain construct
MDSQAQVLILLLLWVSGTCGDIVLTQSPDSLAVSLGERATISCKSSQSLLNSRTRENYLAWYQQKPGQPPKLLIYWASTRESGVPDRFSGSGSGTDFTLTISSLQAEDVAVYYCTQSYNLYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 461 aa, Artificial Sequence
  Description of Artificial Sequence Synthetic heavy construct
MGWNCIIFFLVTTATGVHSQVQLVQSGAEVKKPGSSVKVSCKASGYTFTDYAIQWVRQAPGQGLEWIGVINIYYDNTNYNQKFKGKATMTVDKSTSTAYMELSSLRSEDTAVYYCARAAWYMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAAPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 5 - protein, 467 aa, Artificial Sequence
  Description of Artificial Sequence Synthetic heavy chain construct
MDFGFSLVFLALILKGVQCEVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - protein, 239 aa, Artificial Sequence
  Description of Artificial Sequence Synthetic kappa chain construct
MKLPVRLLVLLLFWIPASRGDVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 1438 aa, Artificial Sequence
  Description of Artificial Sequence Synthetic construct
ATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFLTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY